(19) United States
(12) Patent Application Publication
(10) Pub. No.: US 2021/0213001 A1
(43) Pub. Date: Jul. 15, 2021

(54) METHOD OF TREATING A SLEEP BREATHING DISORDER

(71) Applicants: Ian Cooke, Hawthorn, Victoria (AU); Monash University, Clayton, Victoria (AU)

(72) Inventors: Ian COOKE, Hawthorn (AU); Philip J. Berger, Clayton (AU)

(21) Appl. No.: 17/274,150

(22) PCT Filed: Sep. 6, 2019
(86) PCT No.: PCT/AU2019/050952

§ 371 Date: Mar. 5, 2021

(30) Foreign Application Priority Data
Sep. 6, 2018 (AU) 2018903325

(51) Int. Cl. A61K 31/44; A61K 31/27; A61P 11/16; A61K 31/343; A61K 9/00; A61K 45/06

(52) U.S. Cl. CPC A61K 31/44; A61K 31/27; A61K 45/06; A61K 31/343; A61K 9/0053; A61P 11/16

(57) ABSTRACT

The present disclosure relates to methods of treating or preventing a sleep breathing disorder. More specifically, the disclosure relates to a method of treating or preventing sleep apnea comprising the use of a KCNQ potassium channel opener.

### FIELD

The present disclosure relates to a method of treating or preventing a sleep breathing disorder. More specifically, the disclosure relates to a method of treating or preventing sleep apnea comprising the use of a KCNQ potassium channel opener.

### BACKGROUND

Of the sleep breathing disorders, sleep apnea is a common medical condition, present in an estimated 5% to 9% of the population. The condition is associated with excessive daytime sleepiness and composite risk for cardiovascular morbidity and mortality. In particular, sleep apnea has been shown to exacerbate damage to the myocardium with a net effect of a weakening of the heart and, ultimately, an increase in heart-related death. Furthermore, sleep apneas have also been associated with an increased incidence of metabolic disease, psychiatric disorders, cognitive deterioration, dementia, and Alzheimer's disease, amongst other conditions.

The most common form of sleep apnea in adults is “obstructive sleep apnea”, resulting from the cessation of airflow due to closure of the naso- and/or oro-pharyngeal airway. “Non-obstructive sleep apnea”, also referred to as “central sleep apnea”, occurs when absent airflow is accompanied by an absence in inspiratory efforts. Components of obstructive and non-obstructive apneas frequently coexist in a single event, producing “mixed sleep apnea”.

During wakefulness and sleep, breathing is driven by feedback from sensory receptors (chemoreceptors) that monitor the partial pressure of carbon dioxide (PaCO₂) and oxygen (PaO₂) in the arterial blood, as well as the arterial pressure of carbon dioxide (PCO₂) and H⁺ concentration of the cerebrospinal fluid. The chemoreceptors that monitor PaCO₂ and PaO₂ are located in the carotid bodies, being bilateral structures situated where the common carotid artery divides into internal and external branches. These receptors are referred to as the “peripheral chemoreceptors”. The chemoreceptors that respond to changes in cerebrospinal fluid PCO₂ and H⁺ are located in the floor of the brainstem, and are referred to as the “central chemoreceptors”. While under resting conditions, both the peripheral and central chemoreceptors contribute to ventilatory drive, it is the activity of the peripheral chemoreceptors that dominates the response of the respiratory system when a transient change in breathing occurs. Accordingly, it is the peripheral chemoreceptors that provide the dominant drive for unstable breathing patterns during sleep. In this way, breathing is driven by respiratory control loop gain (LG) in response to chemoreceptive drive. Ultimately, the instability of the respiratory control system, and hence the propensity of an individual to exhibit a sleep breathing disorder, can be reduced by changing one or more of the four factors that contribute to loop gain (i.e., chemosensitivity, arterial pressure of carbon dioxide, lung volume, timing of the lung-chemoreceptor circulatory delay).

While current treatment approaches can have a substantial impact in reducing the incidence of sleep apneas, treatments are either not tolerated or are too intrusive for a significant portion of patients. For example, a current first-line treatment is the use of a continuous positive airway pressure (CPAP) device. The device comprises a mask that is fitted over the nose and/or mouth of a patient during sleep, and maintains a positive flow of air into the airway to keep the passages open. Adapting to sleeping with such an intrusive device can prove difficult for many patients. Other treatment approaches include the use of dental appliances that reposition the lower jaw and tongue; upper airway surgery to remove tissue in the airway; and nasal expiratory positive airway pressure where a disposable valve covers the nostrils. There also exists treatment using hypoglossal nerve stimulation where a stimulator is implanted in the patient's chest with leads connected to the hypoglossal nerve that controls tongue movement, as well as to a breathing sensor. The sensor monitors breathing patterns during sleep and stimulates the hypoglossal nerve to move the tongue to maintain an open airway.

Consequently, therefore, there is a need for a new method of treating or preventing a sleep breathing disorder in a subject. In particular, there is a need for a new method of treating or preventing a sleep breathing disorder in a subject that circumvents the intrusive, current treatment approaches.

### SUMMARY

The present inventors have identified a novel and alternative method of treating or preventing a sleep breathing disorder in a subject.

Accordingly, in one aspect there is provided a method of treating or preventing a sleep breathing disorder in a subject, the method comprising administering an effective amount of a KCNQ potassium channel opener.

In some embodiments, the KCNQ potassium channel opener is selected from a compound of Formula I, II or III:

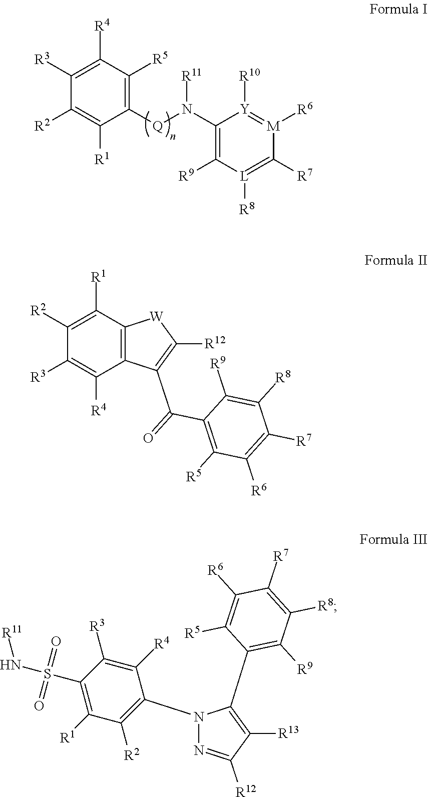

wherein
n is 0, 1 or 2;
   when n is 1, Q is CR¹⁴R¹⁵ or C(O); and when n is 2, Q are each independently CR¹⁴R¹⁵ or C(O);
Y, M and L are each independently C or N;
   when Y is N, R¹⁰ is not present; when M is N, R⁶ is not present; and when L is N, R⁸ is not present;
W is O, S or NH;
R¹, R², R³, R⁴, R⁵, R⁶, R⁷, R⁸, R⁹, R¹⁰, R¹² and R¹³ are each independently selected from hydrogen, halo, CF₃, CN, NO₂, OH, SH, NH₂, S(O)OH, C(O)H, C(O)₂H, OC(O)R¹⁴, C(O)R¹⁴, C(O)NR¹⁴R¹⁵, C(O)OR¹⁴, OR¹⁴, NHC(O)OR¹⁴, OS(O)₂R¹⁴, S(O)₂NR¹⁴R¹⁵, NR¹⁴R¹⁵, SR¹⁴, C₁₋₂₀alkyl-C(O)OR¹⁴, C₁₋₂₀alkyl, C₂₋₂₀alkenyl, C₂₋₂₀alkynyl, monocyclic or polycyclic carbocyclic, and monocyclic or polycyclic heterocyclic;
   wherein R¹⁴ and R¹⁵ are each independently selected from H and C₁₋₁₀alkyl, wherein the C₁₋₁₀alkyl is optionally interrupted with one or more heteroatoms independently selected from O, N and S; and wherein the C₁₋₁₀alkyl is optionally substituted with one or more substituents independently selected from the group consisting of hydrogen, halo, CF₃, CN, NO₂, OH, SH, NH₂, S(O)OH, C(O)H and C(O)₂H; and
   wherein the C₁₋₂₀alkyl, C₂₋₂₀alkenyl, and C₂₋₂₀alkynyl are each optionally interrupted with one or more heteroatoms independently selected from O, N and S; and wherein the C₁₋₂₀alkyl, C₂₋₂₀alkenyl, C₂₋₂₀alkynyl, monocyclic or polycyclic carbocyclic, and monocyclic or polycyclic heterocyclic are each optionally substituted with one or more substituents independently selected from the group consisting of halo, CF₃, CN, NO₂, OH, SH, NH₂, S(O)OH, C(O)H and C(O)₂H; and
R¹¹ is selected from the group consisting of hydrogen and C₁₋₁₀alkyl.

In some embodiments, the KCNQ potassium channel opener is a KCNQ2-5 channel opener.

In some embodiments, the peripheral plasma concentration of the KCNQ potassium channel opener is greater than the central plasma concentration of the KCNQ potassium channel opener.

In some embodiments, the peripheral plasma concentration of the KCNQ potassium channel opener is at least about 1.5 times greater than the central plasma concentration of the KCNQ potassium channel opener.

In some embodiments, the KCNQ potassium channel opener is selected from the group consisting of Retigabine, Flupirtine, SF0034, RL648_81, Diclofenac, Meclofenamic acid, Benzbromarone, Celecoxib, ICA-169673, and Compound 40.

In some embodiments, the KCNQ potassium channel opener is Retigabine.

In some embodiments, the sleep breathing disorder is selected from the group consisting of non-obstructive, or central (CSA), sleep apnea, obstructive sleep apnea (OSA), and mixed sleep apnea.

In some embodiments, the central sleep apnea (CSA) is Cheyne-Stokes respiration (CSR).

In some embodiments, the subject is tested for elevated loop gain prior to or following administration of the KCNQ potassium channel opener.

In some embodiments, the KCNQ potassium channel opener is administered in combination with an additional therapeutic agent that acts in synergy with KCNQ channel openers.

In some embodiments, the additional therapeutic agent is selected from the group consisting of purinergic receptor antagonists, dopamine receptor agonists, alpha-2 adrenergic receptor agonists, GABA_{A} receptor agonists, H₃ antihistamines and modulators of H₂S and CO mediated transduction mechanisms.

In some embodiments, the additional therapeutic agent is a P2X3 receptor antagonist.

In some embodiments, the KCNQ potassium channel opener is administered to the subject orally.

In some embodiments, the KCNQ potassium channel opener is administered as a once-daily dosage of 5 mg to 300 mg.

In some embodiments, the KCNQ potassium channel opener is Retigabine administered in a once-daily dosage of 5 mg to 300 mg.

In some embodiments, the KCNQ potassium channel opener is Flupirtine administered in a once-daily dosage of 100 mg to 200 mg.

In some embodiments, the subject is a human.

In some embodiments, an improvement in sleep apnea in a subject is detected by an increase in arterial PCO₂.

In some embodiments, an improvement in sleep apnea in a subject is detected by an increase in arterial H⁺ concentration.

In some embodiments, an improvement in sleep apnea in a subject is detected by conducting a sleep study.

In some embodiments, the sleep study assesses at least one of the subject's sleep state, eye movement, muscle activity, heart rate, respiratory effort, airflow, blood oxygen levels, arterial PCO₂, and arterial H⁺ concentration.

In another aspect, there is provided a KCNQ potassium channel opener for use in treating or preventing a sleep breathing disorder in a subject.

In another aspect, there is provided an agent selected from a KCNQ potassium channel opener for use in treating or preventing a sleep breathing disorder in a subject.

In another aspect, there is provided the use of a KCNQ potassium channel opener for treating or preventing a sleep breathing disorder in a subject.

In another aspect, there is provided the use of a KCNQ potassium channel opener in the manufacture of a medicament for the treatment or prevention of a sleep breathing disorder in a subject.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1: Effect of Retigabine on periodic breathing induced by transient hyperventilation.

FIG. 1A shows an epoch of periodic breathing induced by transient hyperventilation in the absence of Retigabine in a Domperidone-treated lamb. At the start of the trace (H), the lamb is hyperventilated to achieve hypocapnic conditions and fully saturated arterial blood, with an SaO₂ approximating 100%. After hyperventilation was terminated, there was a period of apnea (A) during which SaO₂ fell to an intended level of approximately 50%. In all animals, apnea was followed by an extended period of periodic breathing (PB) that exhibited the hallmark crescendo/de-crescendo profile of Cheyne-Stokes breathing typical in humans with central sleep anpea. The epoch of periodic breathing illustrated comprised 30 cycles of ventilation and apnea (the first 24 are shown) and extended for 6 minutes; the average value of loop gain across the epoch was 1.71.

FIG. 1B shows an epoch of periodic breathing induced by transient hyperventilation in the same lamb as depicted in FIG. 1A following the administration of Retigabine. In this instance, SaO₂ again fell to approximately 50% before the post-hyperventilation apnea terminated and spontaneous breathing re-commenced. The epoch of periodic breathing lasted only 115 seconds and comprised only 6 cycles of ventilation and apnea. The average value of loop gain across this short epoch of period breathing was 1.24.

FIG. 2: Effect of flupirtine on periodic breathing induced by transient hyperventilation.

FIG. 2A shows an epoch of periodic breathing induced by transient hyperventilation in the absence of Flupirtine. Transient hyperventilation caused an apnea lasting approximately 15 seconds during which SaO₂ fell to 80%. This was followed by an epoch of periodic breathing that lasted 320 seconds and comprised 24 cycles of ventilation and apnea. The average value of loop gain across the epoch was 1.51.

FIG. 2B shows an epoch of periodic breathing induced by transient hyperventilation in the same lamb as depicted in FIG. 2A following the administration of Flupirtine. SaO₂ fell to 55% before the post-hyperventilation apnea terminated and spontaneous breathing re-commenced. The epoch of periodic breathing lasted 130 seconds and comprised 9 cycles of apnea and breathing. The average value of loop gain across this epoch of period breathing was 1.16.

FIG. 3: Summary of effects of Retigabine and Flupirtine on periodic breathing induced by transient hyperventilation.

FIG. 3A shows the average duration of epochs of hyperventilation-induced periodic breathing in each of the four lambs studied, before and after the administration of Retigabine or Flupirtine. Retigabine was administered to lambs 1, 2 and 3. Flupirtine was administered to lamb 4.

FIG. 3B shows the average values of loop gain across two successive epochs of hyperventilation-induced periodic breathing in each of the four lambs studied, before and after the administration of Retigabine or Flupirtine. Retigabine was administered to lambs 1, 2 and 3. Flupirtine was administered to lamb 4.

FIG. 4: Clinical study of the acute effects of a KCNQ channel opener on peripheral chemoreceptor sensitivity and sleep disordered breathing in human subjects.

FIG. 4A shows the protocol for a clinical study to assess the effect of a KCNQ channel opener on peripheral chemoreceptor sensitivity during wakefulness and on sleep architecture, periodic breathing and loop gain during sleep in subjects suffering from sleep disordered breathing.

FIG. 4B shows the detailed protocol for each study night in the clinical study.

FIG. 5: Clinical study of the dose-dependent and duration-dependent effects of a KCNQ channel opener on peripheral chemoreceptor sensitivity and sleep disordered breathing in human subjects.

FIG. 5A shows the protocol for a clinical study to assess the effect of different doses and periods of exposure of a KCNQ channel opener on peripheral chemoreceptor sensitivity during wakefulness and on sleep architecture, periodic breathing and loop gain during sleep in subjects suffering from sleep disordered breathing.

FIG. 5B shows the detailed protocol for each study night in the clinical study.

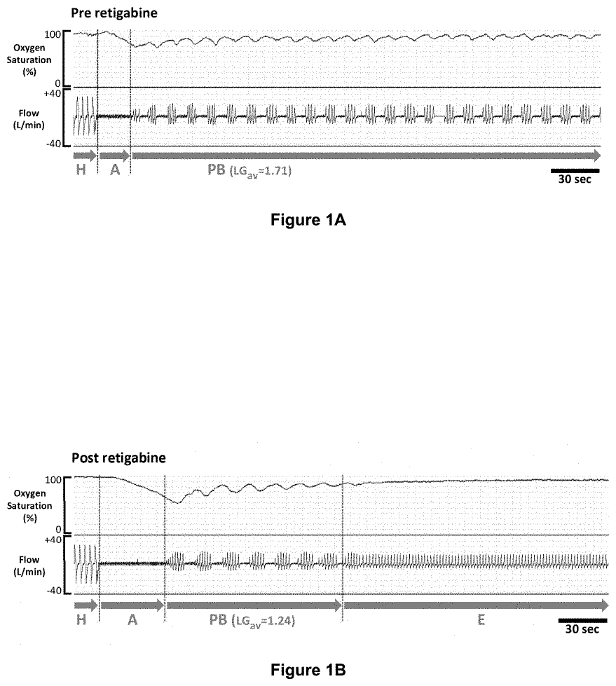

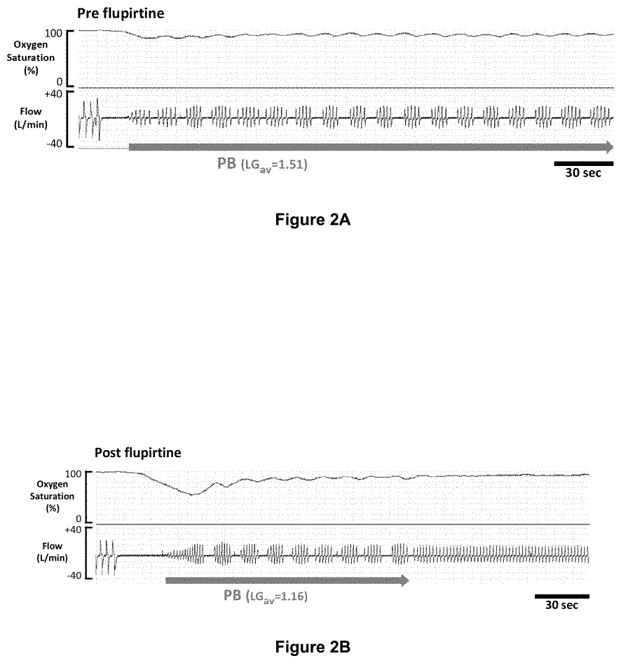

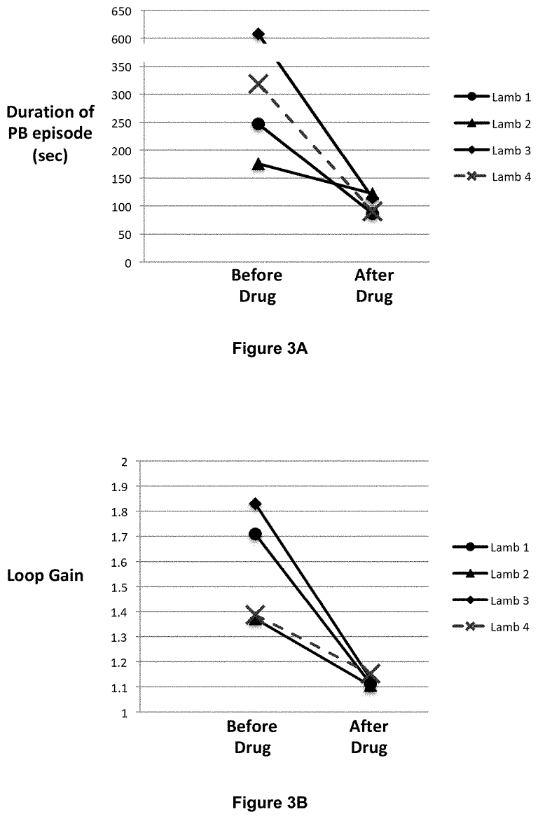

### DETAILED DESCRIPTION

### General Techniques and Definitions

Unless specifically defined otherwise, all technical and scientific terms used herein shall be taken to have the same meaning as commonly understood by one of ordinary skill in the art (e.g., biochemistry, chemistry, medicinal chemistry, physiology, and the like).

As used herein, the term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, of the designated value.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” refers to any organism susceptible to a sleep breathing disorder. In one example, the subject is a mammal. In one example, the subject is human.

As used herein, the term “treating” includes alleviation of the symptoms associated with a specific disorder or condition and eliminating said symptoms. For example, as used herein, the phrase “treating a sleep breathing disorder” refers to alleviating the symptoms associated with a sleep breathing disorder and eliminating said symptoms.

As used herein, the term “prevention” includes prophylaxis of the specific disorder or condition. For example, as used herein, the phrase “preventing a sleep breathing disorder” refers to preventing the onset or duration of the symptoms associated with a sleep breathing disorder.

As would be understood by the person skilled in the art, a KCNQ potassium channel opener would be administered in a therapeutically effective amount. The term “therapeutically effective amount”, as used herein, refers to a KCNQ potassium channel opener being administered in an amount sufficient to alleviate or prevent to some extent one or more of the symptoms of the disorder or condition being treated. The result can be the reduction and/or alleviation of the signs, symptoms, or causes of a disease or condition, or any other desired alteraction of a biological system. In one example, the result may be the reduction in the periodic cessation of breathing (apnea) associated with a sleep breathing disorder. In one example, the result may be the reduction of daytime weariness and/or fatigue associated with a sleep breathing disorder. In one example, the result may be the reduction of the risk of cardiovascular disease associated with a sleep breathing disorder. In one example, the result may be the reduction of the risk of metabolic disease associated with a sleep breathing disorder. In one example, the result may be the reduction of the risk of psychiatric disorders associated with a sleep breathing disorder. In one example, the result may be the reduction of the risk of cognitive deterioration and dementia associated with a sleep breathing disorder. In one example, the result may be the decreased likelihood of the development of Alzheimer's disease.

The term, an “effective amount”, as used herein, refers to an amount of a KCNQ potassium channel opener effective to achieve a desired pharmacologic effect or therapeutic improvement without undue adverse side effects. By way of example only, therapeutically effective amounts may be determined by routine experimentation, including but not limited to a dose escalation clinical trial. The term “therapeutically effective amount” includes, for example, a prophylactically effective amount. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. Thus, it is not always possible to specify an exact “effective amount”. However, an appropriate “effective amount” in any individual case may be determined by one of ordinary skill in the art using routine experimentation. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

The term “onset” of activity, as used herein, refers to the length of time to alleviate or prevent to some extent one or more of the symptoms of the disorder or condition being treated following the administration of a KCNQ potassium channel opener. The term “duration” refers to the length of time that the therapeutic continues to be therapeutically effective, i.e., alleviate or prevent to some extent one or more of the symptoms of the disorder or condition being treated. The person skilled in the art would be aware that onset, peak, and duration of therapy may vary depending on factors such as the patient, the condition of the patient, and the route of administration.

The terms “carbocyclic” and “carbocyclyl” represent a monocyclic or polycyclic ring system wherein the ring atoms are all carbon atoms, e.g., of about 3 to about 20 carbon atoms, and which may be aromatic, non-aromatic, saturated, or unsaturated, and may be substituted and/or contain fused rings. Examples of such groups include aryl groups such as benzene, saturated groups such as cyclopentyl, or fully or partially hydrogenated phenyl, naphthyl and fluorenyl. It will be appreciated that the polycyclic ring system includes bicyclic and tricyclic ring systems.

The terms “heterocyclyl” and “heterocyclic” whether used alone, or in compound words such as heterocyclyloxy, represent a monocyclic or polycyclic ring system wherein the ring atoms are provided by at least two different elements, typically a combination of carbon and one or more of nitrogen, sulfur, and oxygen, although may include other elements for ring atoms such as selenium, boron, phosphorus, bismuth, and silicon, and wherein the ring system is about 3 to about 20 atoms, and which may be aromatic such as a “heteroaryl” group, non-aromatic, saturated, or unsaturated, and may be substituted and/or contain fused rings. For example, the heterocyclyl may be (i) an optionally substituted cycloalkyl or cycloalkenyl group, e.g., of about 3 to about 20 ring members, which may contain one or more heteroatoms such as nitrogen, oxygen, or sulfur (examples include pyrrolidinyl, morpholino, thiomorpholino, or fully or partially hydrogenated thienyl, furyl, pyrrolyl, thiazolyl, oxazolyl, oxazinyl, thiazinyl, pyridyl and azepinyl); (ii) an optionally substituted partially saturated monocyclic or polycyclic ring system in which an aryl (or heteroaryl) ring and a heterocyclic group are fused together to form a cyclic structure (examples include chromanyl, dihydrobenzofuryl and indolinyl); or (iii) an optionally substituted fully or partially saturated polycyclic fused ring system that has one or more bridges (examples include quinuclidinyl and dihydro-1,4-epoxynaphthyl). It will be appreciated that the polycyclic ring system includes bicyclic and tricyclic ring systems.

The term “optionally substituted” means that a functional group is either substituted or unsubstituted, at any available position. Substitution can be with one or more functional groups selected from, e.g., alkyl, alkenyl, alkynyl, cycloalkyl, cycloalkenyl, aryl, heterocyclyl, heteroaryl, formyl, alkanoyl, cycloalkanoyl, aroyl, heteroaroyl, carboxyl, alkoxycarbonyl, cycloalkyloxycarbonyl, aryloxycarbonyl, heterocyclyloxycarbonyl, heteroaryloxycarbonyl, alkylaminocarbonyl, cycloalkylaminocarbonyl, arylaminocarbonyl, heterocyclylaminocarbonyl, heteroarylaminocarbonyl, cyano, alkoxy, cycloalkoxy, aryloxy, heterocyclyloxy, heteroaryloxy, alkanoate, cycloalkanoate, aryloate, heterocyclyloate, heteroaryloate, alkylcarbonylamino, cycloalkylcarbonylamino, arylcarbonylamino, heterocyclylcarbonylamino, heteroarylcarbonylamino, nitro, alkylthio, cycloalkylthio, arylthio, heterocyclylthio, heteroarylthio, alkylsulfonyl, cycloalkylsulfonyl, arylsulfonyl, heterocyclysulfonyl, heteroarylsulfonyl, hydroxyl, halo, haloalkyl, haloaryl, haloheterocyclyl, haloheteroaryl, haloalkoxy, haloalkylsulfonyl, silylalkyl, alkenylsilylalkyl, and alkynylsilylalkyl. It will be appreciated that other groups not specifically described may also be used.

The terms “halo” and “halogen” whether employed alone or in compound words such as haloalkyl, haloalkoxy or haloalkylsulfonyl, represents fluorine, chlorine, bromine or iodine. Further, when used in compound words such as haloalkyl, haloalkoxy or haloalkylsulfonyl, the alkyl may be partially halogenated or fully substituted with halogen atoms which may be independently the same or different. Examples of haloalkyl include, without limitation, —CH₂CH₂F, —CF₂CF₃ and —CH₂CHFCl. Examples of haloalkoxy include, without limitation, —OCHF₂, —OCF₃, —OCH₂CCl₃, —OCH₂CF₃ and —OCH₂CH₂CF₃. Examples of haloalkylsulfonyl include, without limitation, —SO₂CF₃, —SO₂CCl₃, —SO₂CH₂CF₃ and —SO₂CF₂CF₃.

The term “alkyl” whether used alone, or in compound words such as alkoxy, alkylthio, alkylamino, dialkylamino or haloalkyl, represents straight or branched chain hydrocarbons ranging in size from one to about 20 carbon atoms, or more. Thus alkyl moieties include, unless explicitly limited to smaller groups, moieties ranging in size, for example, from one to about 6 carbon atoms or greater, such as, methyl, ethyl, n-propyl, iso-propyl and/or butyl, pentyl, hexyl, and higher isomers, including, e.g., those straight or branched chain hydrocarbons ranging in size from about 6 to about 20 carbon atoms, or greater. In one example, the alkyl moiety is of one to 10 carbon atoms.

The term “alkenyl” whether used alone, or in compound words such as alkenyloxy or haloalkenyl, represents straight or branched chain hydrocarbons containing at least one carbon-carbon double bond, including, unless explicitly limited to smaller groups, moieties ranging in size from two to about 6 carbon atoms or greater, such as, methylene, ethylene, 1-propenyl, 2-propenyl, and/or butenyl, pentenyl, hexenyl, and higher isomers, including, e.g., those straight or branched chain hydrocarbons ranging in size, for example, from about 6 to about 20 carbon atoms, or greater. In one example, the alkenyl moiety is of two to 20 carbon atoms.

The term “alkynyl” whether used alone, or in compound words such as alkynyloxy, represents straight or branched chain hydrocarbons containing at least one carbon-carbon triple bond, including, unless explicitly limited to smaller groups, moieties ranging in size from, e.g., two to about 6 carbon atoms or greater, such as, ethynyl, 1-propynyl, 2-propynyl, and/or butynyl, pentynyl, hexynyl, and higher isomers, including, e.g., those straight or branched chain hydrocarbons ranging in size from, e.g., about 6 to about 20 carbon atoms, or greater. In one example, the alkynyl moiety is of two to 20 carbon atoms.

The term “C₁₋₂₀alkyl,” as used herein refers to a straight chain or branched, saturated or unsaturated hydrocarbon having from 1 to 20 carbon atoms. Representative “C₁₋₂₀alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₂-C₂₀ alkyls (i.e., “C₂₋₂₀alkenyl and C₂₋₂₀alkynyl”) include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl, methyl, ethyl, propyl, isopropyl, n-butyl, isobutyl, sec-butyl, tert-butyl, n-pentyl, isopentyl, neopentyl, n-hexyl, isohexyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₂₀ alkyl, —O—(C₁-C₂₀ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

“Substituted alkyl”, “substituted aryl”, and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO₃⁻, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₂₀ alkyl, C₆-C₂₀ aryl, C₃-C₁₄ heterocycle, protecting group or prodrug moiety. Alkylene, alkenylene, and alkynylene groups as described above may also be similarly substituted.

The terms “carbocyclic” and “carbocyclyl” represent a monocyclic or polycyclic ring system wherein the ring atoms are all carbon atoms, e.g., of about 3 to about 20 carbon atoms, and which may be aromatic, non-aromatic, saturated, or unsaturated, and may be substituted and/or contain fused rings. Examples of such groups include aryl groups such as benzene, saturated groups such as cyclopentyl, or fully or partially hydrogenated phenyl, naphthyl and fluorenyl. It will be appreciated that the polycyclic ring system includes bicyclic and tricyclic ring systems.

The terms “heterocyclyl” and “heterocyclic” whether used alone, or in compound words such as heterocyclyloxy, represents a monocyclic or polycyclic ring system wherein the ring atoms are provided by at least two different elements, typically a combination of carbon and one or more of nitrogen, sulfur and oxygen, although may include other elements for ring atoms such as selenium, boron, phosphorus, bismuth, and silicon, and wherein the ring system is about 3 to about 20 atoms, and which may be aromatic such as a “heteroaryl” group, non-aromatic, saturated, or unsaturated, and may be substituted and/or contain fused rings. For example, the heterocyclyl may be (i) an optionally substituted cycloalkyl or cycloalkenyl group, e.g., of about 3 to about 20 ring members, which may contain one or more heteroatoms such as nitrogen, oxygen, or sulfur (examples include pyrrolidinyl, morpholino, thiomorpholino, or fully or partially hydrogenated thienyl, furyl, pyrrolyl, thiazolyl, oxazolyl, oxazinyl, thiazinyl, pyridyl and azepinyl); (ii) an optionally substituted partially saturated monocyclic or polycyclic ring system in which an aryl (or heteroaryl) ring and a heterocyclic group are fused together to form a cyclic structure (examples include chromanyl, dihydrobenzofuryl and indolinyl); or (iii) an optionally substituted fully or partially saturated polycyclic fused ring system that has one or more bridges (examples include quinuclidinyl and dihydro-1,4-epoxynaphthyl). It will be appreciated that the polycyclic ring system includes bicyclic and tricyclic ring systems.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

The phrase “pharmaceutically acceptable salt”, as used herein, refers to pharmaceutically acceptable organic or inorganic salts of an Exemplary Compound or Exemplary Conjugate. The Exemplary Compounds and Exemplary Conjugates contain at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The terms “pharmaceutically acceptable solvate” and “solvate” refer to an association of one or more solvent molecules and a compound of the invention, e.g., an Exemplary Compound or Exemplary Conjugate. Examples of solvents that form pharmaceutically acceptable solvates include, but are not limited to, water, isopropanol, ethanol, methanol, DMSO, ethyl acetate, acetic acid, and ethanolamine.

### Loop Gain

In the context of cyclic sleep breathing disorders, loop gain is the magnitude of the ventilatory response of the respiratory control system to a sinusoidal respiratory disturbance at the frequency of the cycle. Feedback loops with a value of loop gain that exceeds 1.0 (i.e., response is greater than disturbance) are instrinsically unstable and periodic oscillations in breathing inevitably occur. Conversely, feedback loops with a loop gain value of less than 1.0 (i.e., response is less than disturbance), exhibit transient oscillations that are progressively attenuated and temporary. In a period of hyperventilation (disturbance), a reduction in arterial partial pressure of CO₂ results, which is sensed by the peripheral chemoreceptors after a circulatory delay, which in turn elicits a later reduction in ventilator drive (response). In an unstable system, the decrease in ventilatory drive causes a greater degree of hypoventilation compared to the original hyperventilatory disturbance. Oscillations are thus amplified and self-sustained with no initiating factor required.

Four primary factors contribute to respiratory control loop gain, according to the relationship:

$$Loop Gain = G \frac{P a C O 2 - P i C O 2}{Lung Volume} T \qquad Eqn 1$$

where G is the chemosensitivity, defined as the change in ventilation in response to a unit change in PaCO₂; PaCO₂ is the arterial partial pressure of CO₂; PiCO₂ is inspired CO₂; Lung Volume is the end-expiratory lung volume (e.g., functional residual capacity); and T is a timing factor that incorporates the lung-chemoreceptor circulatory delay. In one example, chemosensitivity contributes to respiratory loop gain. In one example, the arterial pressure of CO₂ contributes to respiratory loop gain. In one example, lung volume contributes to respiratory loop gain. In one example, the timing of the lung-chemoreceptor circulatory delay contributes to respiratory loop gain. In one example, a combination of any two or more of chemosensitivity, the arterial pressure of CO₂, lung volume, and the timing of the lung-chemoreceptor circulatory delay contributes to respiratory loop gain.

### Sleep Breathing Disorders

Sleep breathing disorders include a number of disorders that result in the abnormal breathing of a subject during sleep. One of the most common sleep breathing disorders is sleep apnea. In one example, the sleep breathing disorder is sleep apnea. Sleep apnea itself may be classified as “obstructive sleep apnea”, “non-obstructive sleep apnea” or “central sleep apnea”, and “mixed sleep apnea”. In one example, the sleep breathing disorder is obstructive sleep apnea. In one example, the sleep breathing disorder is non-obstructive, or central, sleep apnea. In one example, the sleep breathing disorder is mixed sleep apnea. In one example, the sleep apnea is obstructive sleep apnea. In one example, the sleep apnea is non-obstructive or central sleep apnea. In one example, the sleep apnea is mixed sleep apnea. One type of non-obstructive, or central, sleep apnea is Cheyne-Stokes respiration. In one example, the sleep breathing disorder is Cheyne-Stokes respiration. In one example, the sleep apnea is Cheyne-Stokes respiration. There also exists additional types of sleep breathing conditions, including altitude-based central sleep apnea, periodic breathing or apnea of a newborn and/or infant, and idiopathic central sleep apnea. In one example, the sleep breathing condition is altitude-based central sleep apnea. In one example, the sleep breathing condition is periodic breathing or apnea of a newborn and/or infant. In one example, the sleep breathing disorder is idiopathic central sleep apnea.

### KCNQ Potassium Channel Opener

Voltage-gated potassium channels (VGKCs) are transmembrane channels specific for potassium and sensitive to voltage changes in the membrane potential of a cell. During an action potential, VGKCs play a crucial role in returning a depolarised cell to a resting state. The VGKCs are classified into 12 classes, which includes the Kᵥ7 (KCNQ) family. Neuronal KCNQ channels are responsible for controlling neuronal excitability by regulating potassium conductance across the cell membrane. When the channel is open, potassium is allowed to flow into a neuron (hyperpolarising current) to oppose excitation (depolarisation of the membrane).

The hyperpolarising potassium current that flows through the Kᵥ7 channels is termed the “M current”. It has been found that activating the M current reduces loop gain, and therefore provides a potential means of treating and/or preventing sleep breathing disorders. In one example, activation of the M current reduces loop gain. In one example, activation of the M current results in a reduction of sleep breathing disorders. In one example, activation of the M current results in a reduction of sleep apnea. In one example, activation of the M current provides a method of treating a sleep breathing disorder. In one example, activation of the M current provides a method of treating a sleep apnea.

There are molecules, or ligands, which are known to open KCNQ potassium channels. As used herein, the term “KCNQ potassium channel opener” refers to an agent capable of opening a KCNQ potassium channel, so as to allow the influx of potassium (e.g., M current activation). The term “KCNQ potassium channel opener” is used interchangeably with the term “KCNQ potassium channel activator”. Such molecules known to open KCNQ potassium channels are often small molecules. As used herein, the term “small molecule” refers to an organic molecule with a molecule weight of generally less than 900 Daltons. Larger molecules such as, for example, nucleic acids, proteins and polysaccharides, are typically not considered small molecules. The person skilled in the art would appreciate that organic small molecules are particularly useful as therapeutic agents. In one example, the KCNQ potassium channel opener is a small molecule. In one example, the KCNQ potassium channel opener has a molecule weight of less than about 900 Daltons.

The Kᵥ7 family is further classified into five subtypes, namely Kᵥ7.1 (KCNQ1), Kᵥ7.2 (KCNQ2), Kᵥ7.3 (KCNQ3), Kᵥ7.4 (KCNQ4), and Kᵥ7.5 (KCNQ5). Functional potassium channels are formed as tetramers of these subtypes, either as homomers or heteromers. For example, Kᵥ7.2/3 (KCNQ2/3) channels are formed as heteromers of two Kᵥ7.2 (KCNQ2) subunits and two Kᵥ7.3 (KCNQ3) subunits. The KCNQ potassium channel opener may activate a single potassium channel. In one example, the KCNQ potassium channel opener is a Kᵥ7.1 (KCNQ1) channel opener. In one example, the KCNQ potassium channel opener is a Kᵥ7.2 (KCNQ2) channel opener. In one example, the KCNQ potassium channel opener is a Kᵥ7.3 (KCNQ3) channel opener. In one example, the KCNQ potassium channel opener is a Kᵥ7.4 (KCNQ4) channel opener. In one example, the KCNQ potassium channel opener is a Kᵥ7.5 (KCNQ5) channel opener. Alternatively, the KCNQ potassium channel opener may activate two or more potassium channels. In one example, the KCNQ potassium channel opener is a Kᵥ7.2 (KCNQ2) and Kᵥ7.5 (KCNQ5) channel opener. In one example, the KCNQ potassium channel opener is a Kᵥ7.2 (KCNQ2), Kᵥ7.3 (KCNQ3), Kᵥ7.4 (KCNQ4), and Kᵥ7.5 (KCNQ5) channel opener.

Furthermore, KCNQ potassium channels are located in both the peripheral nervous system (peripheral chemoreceptors) and central nervous system (central chemoreceptors). It is therefore appreciated that a KCNQ potassium channel opener must cross the blood brain barrier (BBB) to interact with a central chemoreceptor. As the ability of a KCNQ potassium channel opener to cross the blood brain barrier is largely dependent on the properties of the molecule itself, such as, for example, molecular weight, lipophilicity, and polar surface area, it follows that different KCNQ potassium channel opener molecules will cross the blood brain barrier to a different extent. Accordingly, different KCNQ potassium channel openers will preferentially distribute to either the peripheral nervous system (and therefore preferentially target peripheral chemoreceptors) or the central nervous system (and therefore preferentially target central chemoreceptors), based on the extent to which the KCNQ potassium channel opener is able to cross the blood brain barrier. The concentration of the KCNQ potassium channel opener in either the peripheral or central nervous system is referred to as the peripheral plasma concentration and central plasma concentration, respectively. In some embodiments, it is preferred that the KCNQ potassium channel opener has a higher peripheral plasma concentration than central plasma concentration (i.e. the KCNQ potassium channel opener preferentially resides in the peripheral nervous system to act preferentially upon peripheral chemoreceptors). In some embodiments, it is preferred that the KCNQ potassium channel opener crosses the blood brain barrier only to such an extent that the KCNQ potassium channel opener maintains a higher peripheral plasma concentration than central plasma concentration. Although, in some embodiments, it may be preferred that the KCNQ potassium channel opener resides in the peripheral nervous system so as to act preferentially upon peripheral chemoreceptors, it will be appreciated that some KCNQ potassium channel opener may cross the blood brain barrier. In one example, the peripheral plasma concentration of the KCNQ potassium channel opener is greater than the central plasma concentration of the KCNQ potassium channel opener. In one example, the peripheral plasma concentration of the KCNQ potassium channel opener is at least about 1.5, 2, 2.5, 3, 3.5, 4, 4.5, 5, 10, 15, 20, or 50 times greater than the central plasma concentration of the KCNQ potassium channel opener. In one example, the peripheral plasma concentration of the KCNQ potassium channel opener is between about 1.5 and 50, 2 and 20, 2.5 and 15, 3 and 10, or 3.5 and 5 times greater than the central plasma concentration of the KCNQ potassium channel opener. In one example, the central plasma concentration of the KCNQ potassium channel opener is less than that of the peripheral plasma concentration of the KCNQ potassium channel opener. In one example, there is provided a method of treating a sleep breathing disorder in a subject, the method comprising administering to the subject a KCNQ potassium channel opener wherein the peripheral plasma concentration of the KCNQ potassium channel opener is greater than the central plasma concentration of the KCNQ potassium channel opener. In one example, a KCNQ potassium channel opener that is largely restricted to the peripheral nervous system is administered to a subject in a method of treating a sleep breathing disorder. In one example, a KCNQ potassium channel opener with relatively poor blood brain barrier penetration is administered to a subject in a method of treating a sleep breathing disorder. In one example, the KCNQ potassium channel opener does not cross the blood brain barrier (i.e. the KCNQ potassium channel opener resides in the peripheral nervous system). In one example, the KCNQ potassium channel opener does not enter the central nervous system.

In some embodiments, the KCNQ potassium channel opener has a structure according to Formula I:

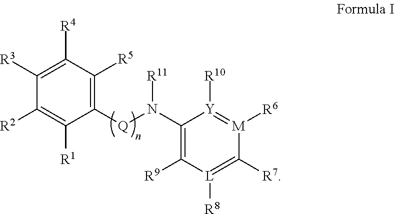

The above compounds of Formula I may be further described as follows.

According to a compound of Formula I, n is 0, 1 or 2. It will be appreciated that when n is 0, N(R¹¹) is directly bonded to the adjacent phenyl ring (e.g., —N(R¹¹)-phenyl):

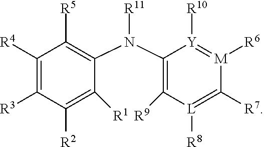

In one example, n is 0. In one example, n is 1. In one example, n is 2. When n is 1, Q is CR¹⁴R¹⁵ or C(O). When n is 2, two Q groups are present (e.g., —N(R¹¹)-Q-Q-):

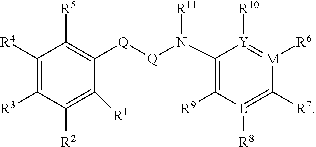

According to a compound of Formula I, each Q is independently CR¹⁴R¹⁵ or C(O). In one example, n is 2, one Q group is CR¹⁴R¹⁵ and the other Q group is C(O):

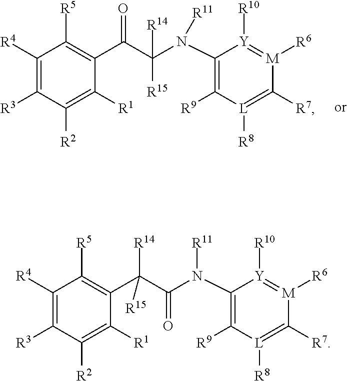

In one example, n is 2, one Q group is CR¹⁴R¹⁵ and the other Q group is CR¹⁴R¹⁵:

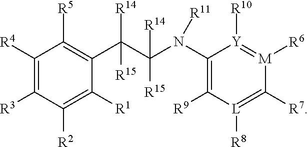

According to a compound of Formula I, Y, M and L are each independently C or N. It will be appreciated that when Y is N, the corresponding R substituent (i.e., R¹⁰) is not present. Similarly, it will be appreciated that when M is N, the corresponding R substituent (ie., R⁶) is not present. Similarly, it will be appreciated that when L is N, the corresponding R substituent (i.e., R⁸) is not present. Accordingly, a compound of Formula I may be selected from the group consisting of the following structures:

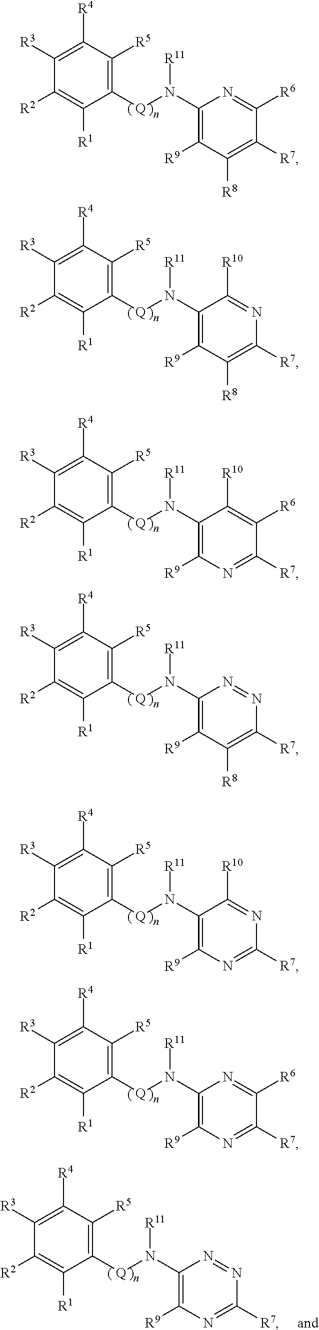

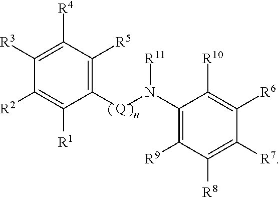

According to a compound of Formula I, R¹, R², R³, R⁴, R⁵, R⁶, R⁷, R⁸, R⁹, R¹⁰, R¹² and R¹³ are each independently selected from the group consisting of hydrogen, halo, CF₃, CN, NO₂, OH, SH, NH₂, S(O)OH, C(O)H, C(O)₂H, OC(O)R¹⁴, C(O)R¹⁴, C(O)NR¹⁴R¹⁵, C(O)OR¹⁴, OR¹⁴, NHC(O)OR¹⁴, OS(O)₂R¹⁴, S(O)₂NR¹⁴R¹⁵, NR¹⁴R¹⁵, SR¹⁴, C₁₋₂₀alkyl-C(O)OR¹⁴, C₂₋₂₀alkenyl, C₂₋₂₀alkynyl, monocyclic or polycyclic carbocyclic, and monocyclic or polycyclic heterocyclic.

According to a compound of Formula I, R¹⁴ and R¹⁵ are each independently selected from hydrogen and C₁₋₁₀alkyl. The C₁₋₁₀alkyl is optionally interrupted with one or more heteroatoms independently selected from O, N and S, and the C₁₋₁₀alkyl is optionally substituted with one or more substituents independently selected from the group consisting of hydrogen, halo, CF₃, CN, NO₂, OH, SH, NH₂, S(O)OH, C(O)H and C(O)₂H.

According to a compound of Formula I, the C₁₋₂₀alkyl, C₂₋₂₀alkenyl, and C₂₋₂₀alkynyl are each optionally interrupted with one or more heteroatoms independently selected from O, N and S, and the C₁₋₂₀alkyl, C₂₋₂₀alkenyl, C₂₋₂₀alkynyl, monocyclic or polycyclic carbocyclic, and monocyclic or polycyclic heterocyclic are each optionally substituted with one or more substituents independently selected from the group consisting of halo, CF₃, CN, NO₂, OH, SH, NH₂, S(O)OH, C(O)H and C(O)₂H.

According to a compound of Formula I, R¹¹ is selected from the group consisting of hydrogen and C₁₋₁₀alkyl.

In one example, the KCNQ potassium channel opener according to Formula I is Retigabine:

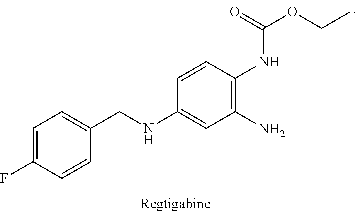 [SMILES: CCOC(=O)Nc1ccc(NCc2ccc(F)cc2)cc1N]

In one example, the KCNQ potassium channel opener according to Formula I is Flupirtine:

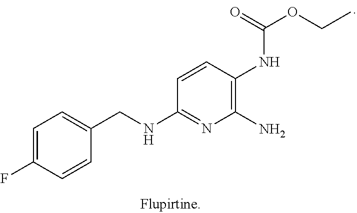 [SMILES: CCOC(=O)Nc1ccc(NCc2ccc(F)cc2)nc1N]

In one example, the KCNQ potassium channel opener according to Formula I is SF0034:

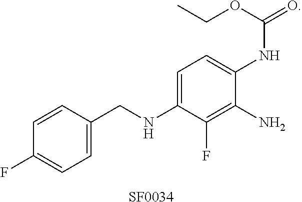

In one example, the KCNQ potassium channel opener according to Formula I is RL648_81:

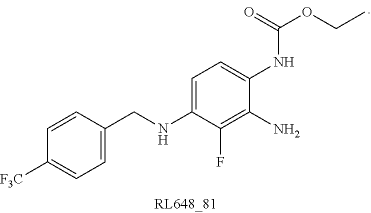 [SMILES: CCOC(=O)Nc1ccc(NCc2ccc(C(F)(F)F)cc2)c(F)c1N]

In one example, the KCNQ potassium channel opener according to Formula I is Diclofenac:

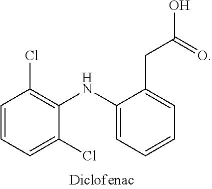 [SMILES: O=C(O)Cc1ccccc1Nc1c(Cl)cccc1Cl]

In one example, the KCNQ potassium channel opener according to Formula I is Meclofenamic acid:

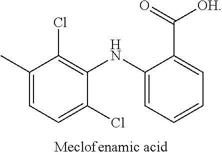 [SMILES: Cc1ccc(Cl)c(Nc2ccccc2C(=O)O)c1Cl]

In one example, the KCNQ potassium channel opener according to Formula I is ICA-069673:

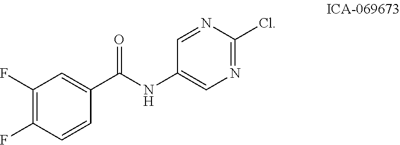 [SMILES: O=C(Nc1cnc(Cl)nc1)c1ccc(F)c(F)c1]

In one example, the KCNQ potassium channel opener according to Formula I is Compound 40:

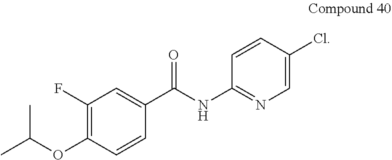 [SMILES: CC(C)Oc1ccc(C(=O)Nc2ccc(Cl)cn2)cc1F]

In some embodiments, the KCNQ potassium channel opener has a structure according to Formula II:

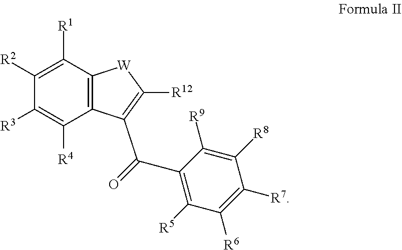

The above compounds of Formula II may be further described as follows.

According to a compound of Formula II, W is O, S or NH. In one example, W is O. In one example, W is S. In one example, W is NH. Accordingly, a compound of Formula II may be selected from the group consisting of the following structures:

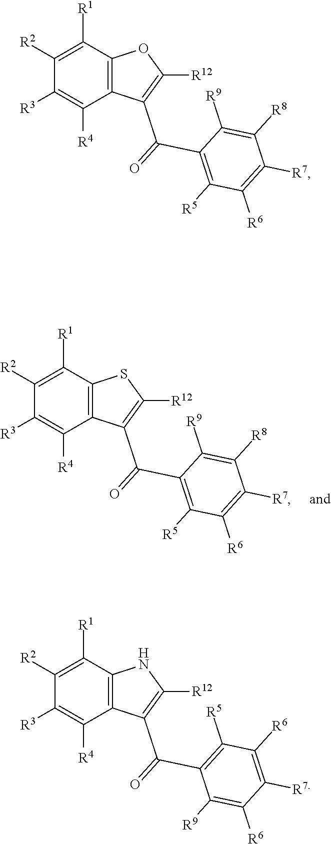

According to a compound of Formula II, R¹, R², R³, R⁴, R⁵, R⁶, R⁷, R⁸, R⁹ and R¹² are each independently selected from the group consisting of hydrogen, halo, CF₃, CN, NO₂, OH, SH, NH₂, S(O)OH, C(O)H, C(O)₂H, OC(O)R¹⁴, C(O)R¹⁴, C(O)NR¹⁴R¹⁵, C(O)OR¹⁴, OR¹⁴, NHC(O)OR¹⁴, OS(O)₂R¹⁴, S(O)₂NR₁₄R¹⁵, NR¹⁴R¹⁵, SR¹⁴, C₁₋₂₀alkyl-C(O)OR¹⁴, C₁₋₂₀alkyl, C₂₋₂₀alkenyl, C₂₋₂₀alkynyl, monocyclic or polycyclic carbocyclic, and monocyclic or polycyclic heterocyclic.

According to a compound of Formula II, R¹⁴ and R¹⁵ are each independently selected from hydrogen and C₁₋₁₀alkyl. The C₁₋₁₀alkyl is optionally interrupted with one or more heteroatoms independently selected from O, N and S, and the C₁₋₁₀alkyl is optionally substituted with one or more substituents independently selected from the group consisting of hydrogen, halo, CF₃, CN, NO₂, OH, SH, NH₂, S(O)OH, C(O)H and C(O)₂H.

According to a compound of Formula II, the C₁₋₂₀alkyl, C₂₋₂₀alkenyl, and C₂₋₂₀alkynyl are each optionally interrupted with one or more heteroatoms independently selected from O, N and S, and the C₁₋₂₀alkyl, C₂₋₂₀alkenyl, C₂₋₂₀alkynyl, monocyclic or polycyclic carbocyclic, and monocyclic or polycyclic heterocyclic are each optionally substituted with one or more substituents independently selected from the group consisting of halo, CF₃, CN, NO₂, OH, SH, NH₂, S(O)OH, C(O)H and C(O)₂H.

In one example, the KCNQ potassium channel opener according to Formula II is Benzbromarone:

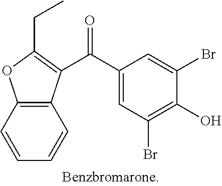 [SMILES: CCc1oc2ccccc2c1C(=O)c1cc(Br)c(O)c(Br)c1]

In some embodiments, the KCNQ potassium channel opener has a structure according to Formula III:

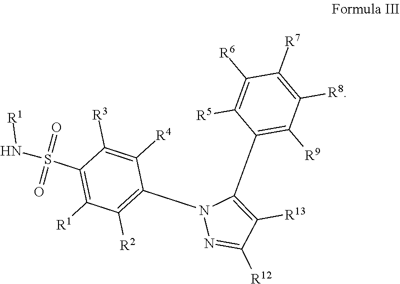

The above compounds of Formula III may be further described as follows.

According to a compound of Formula III, R¹, R², R³, R⁴, R⁵, R⁶, R⁷, R⁸, R⁹, R¹² and R¹³ are each independently selected from the group consisting of hydrogen, halo, CF₃, CN, NO₂, OH, SH, NH₂, S(O)OH, C(O)H, C(O)₂H, OC(O)R¹⁴, C(O)R¹⁴, C(O)NR¹⁴R¹⁵, C(O)OR¹⁴, OR¹⁴, NHC(O)OR¹⁴, OS(O)₂R¹⁴, S(O)₂NR¹⁴R¹⁵, NR¹⁴R¹⁵, SR¹⁴, C₁₋₂₀alkyl-C(O)OR¹⁴, C₁₋₂₀alkyl, C₂₋₂₀alkenyl, C₂₋₂₀alkynyl, monocyclic or polycyclic carbocyclic, and monocyclic or polycyclic heterocyclic.

According to a compound of Formula III, R¹⁴ and R¹⁵ are each independently selected from hydrogen and C₁₋₁₀alkyl. The C₁₋₁₀alkyl is optionally interrupted with one or more heteroatoms independently selected from O, N and S, and the C₁₋₁₀alkyl is optionally substituted with one or more substituents independently selected from the group consisting of hydrogen, halo, CF₃, CN, NO₂, OH, SH, NH₂, S(O)OH, C(O)H and C(O)₂H.

According to a compound of Formula III, the C₁₋₂₀alkyl, C₂₋₂₀alkenyl, and C₂₋₂₀alkynyl are each optionally interrupted with one or more heteroatoms independently selected from O, N and S, and the C₁₋₂₀alkyl, C₂₋₂₀alkenyl, C₂₋₂₀alkynyl, monocyclic or polycyclic carbocyclic, and monocyclic or polycyclic heterocyclic are each optionally substituted with one or more substituents independently selected from the group consisting of halo, CF₃, CN, NO₂, OH, SH, NH₂, S(O)OH, C(O)H and C(O)₂H.

According to a compound of Formula III, R¹¹ is selected from the group consisting of hydrogen and C₁₋₁₀alkyl.

In one example, the KCNQ potassium channel opener according to Formula III is Celecoxib:

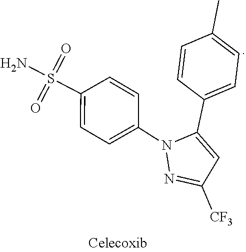 [SMILES: Cc1ccc(-c2cc(C(F)(F)F)nn2-c2ccc(S(N)(=O)=O)cc2)cc1]

It will be appreciated that any of the optional heteroatoms or substituents referred to above in Formula I, II, or III, with reference to “one or more”, unless otherwise stated, may be any integer such as 1, 2, 3, 4, 5, 6, etc., or for example a range of 1 to 6 substituents, 1 to 3 substituents, or 1 to 2 substituents.

### Method of Treatment

It has been surprisingly found that a KCNQ channel opener, which activates the M current, results in a reduction in loop gain, and therefore provides a potential means of treating and/or preventing sleep breathing disorders. In some embodiments, there is provided a method of treating or preventing a sleep breathing disorder in a subject, the method comprising administering an effective amount of a KCNQ potassium channel opener, wherein the KCNQ potassium channel opener activates the M current and reduces loop gain. In one example, there is provided a method of treating or preventing a sleep breathing disorder in a subject, the method comprising administering an effective amount of a KCNQ potassium channel opener. In one example, there is provided a method of treating or preventing a sleep apnea in a subject, the method comprising administering an effective amount of a KCNQ potassium channel opener. In one example, there is provided a method of treating or preventing non-obstructive, or central, sleep apnea in a subject, the method comprising administering an effective amount of a KCNQ potassium channel opener. In one example, there is provided a method of treating or preventing obstructive sleep apnea in a subject, the method comprising administering an effective amount of a KCNQ potassium channel opener. In one example, there is provided a method of treating or preventing mixed sleep apnea in a subject, the method comprising administering an effective amount of a KCNQ potassium channel opener. In one example, there is provided a method of treating or preventing Cheyne-Stokes respiration in a subject, the method comprising administering an effective amount of a KCNQ potassium channel opener. In one example, there is provided a method of treating or preventing altitude-based central sleep apnea in a subject, the method comprising administering an effective amount of a KCNQ potassium channel opener. In one example, there is provided a method of treating or preventing periodic breathing or apnea of a newborn and/or infant, the method comprising administering an effective amount of a KCNQ potassium channel opener. In one example, there is provided a method of treating or preventing idiopathic central sleep apnea in a subject, the method comprising administering an effective amount of a KCNQ potassium channel opener.

### Pre-Existing or Other Health Conditions

In some embodiments, the subject may or may not have pre-existing health conditions additional to a sleep breathing disorder. The Kᵥ7 family is known to be implicated in epilepsy, chronic and neuropathic pain, deafness, and mental illness. Consequently, KCNQ potassium channel openers are administered in the treatment of such diseases, including, for example, epilepsy, acute and chronic pain, inflammation, arthritis including osteoarthritis and rheumatoid arthritis, dysmenorrhea, and gout.

Such pre-existing health conditions may or may not be associated with a sleep breathing disorder. That is, a subject may suffer from a sleep breathing disorder independently of suffering from a pre-existing health condition, or alternatively the subject may be suffering from a pre-existing health condition that is associated with a sleep breathing disorder. In one example, the subject having the sleep breathing disorder is not suffering from a pre-existing condition. In one example, the subject having the sleep breathing disorder is suffering from a pre-existing condition.

The pre-existing health condition may or may not have been diagnosed by a medical practitioner. That is, the subject having the sleep breathing disorder may be suffering from an undiagnosed pre-existing health condition. In one example, the subject having the sleep breathing disorder is suffering from a diagnosed pre-existing condition. In one example, the subject having the sleep breathing disorder is suffering from an undiagnosed pre-existing condition.

It is possible that a subject is being administered with a KCNQ potassium channel opener for the treatment of a condition associated with the Kᵥ7 family (e.g., epilepsy, acute and chronic pain, inflammation, arthritis including osteoarthritis and rheumatoid arthritis, dysmenorrhea, and/or gout) at the time of suffering from a sleep breathing disorder. Otherwise, it is possible that a subject is not suffering from a condition associated with the Kᵥ7 family (e.g., epilepsy, acute and chronic pain, inflammation, arthritis including osteoarthritis and rheumatoid arthritis, dysmenorrhea, and/or gout) at the time of suffering from a sleep breathing disorder. In one example, the subject is not being treated for a condition associated with the Kᵥ7 family (e.g., epilepsy, acute and chronic pain, inflammation, arthritis including osteoarthritis and rheumatoid arthritis, dysmenorrhea, and/or gout). In one example, the subject is not being treated for epilepsy. In one example, the subject is not being treated for a developmental disorder. A developmental disorder includes, but is not limited to, Autistic Spectrum Disorder, Pervasive Developmental Disorder, Autism, Angelman Syndrome, Fragile X Syndrome, Fragile X-associated Tremor/Ataxia Syndrome (FXTAS), Rett Syndrome, Asperger's Syndrome, Childhood Disintegrative Disorder, Attention-Deficit/Hyperactivity Disorder (ADHD), Prader-Willi Syndrome, Landau-Kleffner Syndrome, Rasmussen's Syndrome, Dravet Syndrome, Tardive Dyskinesia, William Syndrome, seizure disorders, and seizure disorders associated with any of the foregoing developmental disorders. In one example, the subject is not being treated for a neurodegenerative disease. A neurodegenerative disorder includes, but is not limited to, Alzheimer's Disease, Amyotrophic lateral sclerosis, Friedreich's ataxia, Huntington's, Lewy body disease, Parkinson's disease, and spinal muscular atrophy. Examples of seizure disorders include, but are not limited to, epilepsy, epilepsy with generalised tonic-clonic seizures, epilepsy with myoclonic abcences, frontal lobe epilepsy, temporal lobe epilepsy, Landau-Kleffner Syndrome, Dravet Syndrome, Rasmussen's Syndrome, Doose Syndrome, CDKL5 disorder, West syndrome, Lennox-Gastaut Syndrome (LGS), Rett Syndrome, Ohtahara Syndrome, essential tremor, acute repetitive seiures, benign rolandic epilepsy, status epilepticus, refractory status epilepticus, super-refractory status epilepticus, PCDH19 pediatric epilepsy, increased seizure activity, and breakthrough seizures. In one example, the subject is not being treated for acute and/or chronic pain. In one example, the subject is not being treated for inflammation. In one example, the subject is not being treated for arthritis including osteoarthritis and rheumatoid arthritis. In one example, the subject is not being treated for dysmenorrhea. In one example, the subject is not being treated for gout.

### Administration

In some embodiments, the KCNQ potassium channel opener is administered to the subject by various routes, e.g., oral, topical, subcutaneous, transdermal, intramuscular, intravenous, or intraperitoneal. Several KCNQ potassium channel openers (e.g., Retigabine, Flupirtine, Diclofenac, Meclofenamic acid, Benzbromarone, Celecoxib) are marketed for oral delivery. It is therefore appreciated that such known drugs exhibit the appropriate properties, i.e., pharmacokinetic and physicochemical properties, to be biopharmaceutically active upon oral administration. In one example, the KCNQ potassium channel opener is administered to the subject orally. In one example, the KCNQ potassium channel opener is administered to the subject intravenously. In one example, the KCNQ potassium channel opener is administered to the subject intramuscularly.

Similarly, such known drugs are prescribed in known dosage amounts for those particular previously known indications. For example, the KCNQ potassium channel opener, Retigabine, is prescribed for the treatment of partial epilepsies and is available as 50 mg, 100 mg, 200 mg, 300 mg, and 400 mg oral dosage amounts. In the treatment of a sleep breathing disorder, the KCNQ potassium channel opener may be administered in a dosage amount prescribed for the treatment of its previously known indication, e.g., Retigabine in the treatment of partial epilepsies, or may be administered in a dosage amount that differs from the amount prescribed for the treatment of its previously known indication. That is, the dosage amount of the KCNQ potassium channel opener required to be administered for the treatment of a sleep breathing disorder is independent of the dosage amount prescribed for the treatment of its previously known indication. In one example, the amount of the KCNQ potassium channel opener required to be administered for the treatment of a sleep breathing disorder is independent of the dosage amount prescribed for the treatment of its previously known indication.

Similarly, such known drugs are prescribed in known dosage regimes. For example, the KCNQ potassium channel opener, Retigabine, is prescribed for the treatment of partial epilepsy initially as a 300 mg daily dose (100 mg three times daily), and may be increased to a 1200 mg daily dose (400 mg three times daily).

In the treatment of a sleep breathing disorder, the KCNQ potassium channel opener may be administered according to a dosage regime prescribed for the treatment of its previously known indication, e.g., Retigabine in the treatment of partial epilepsy, or may be administered according to a different dosage regime than that prescribed for the treatment of its previously known indication. That is, the dosage regime of the KCNQ potassium channel opener required to be administered for the treatment of a sleep breathing disorder is independent of the dosage regime prescribed for the treatment of its previously known indication. In one example, the dosage regime of the KCNQ potassium channel opener administered for the treatment of a sleep breathing disorder is independent of the dosage regime prescribed for the treatment of its previously known indication. In one example, the KCNQ potassium channel opener is administered to a subject as a maximum daily dosage of about 1 mg, 5 mg, 10 mg, 20 mg, 30 mg, 40 mg, 50 mg, 100 mg, 150 mg, 200 mg, 300 mg, 400 mg, 500 mg, 1000 mg, 2000 mg, or 10000 mg for the treatment of a sleep breathing disorder. In one example, the KCNQ potassium channel opener is administered to a subject as a maximum daily dosage of at least 1 mg, 5 mg, 10 mg, 20 mg, 30 mg, 40 mg, 50 mg, 100 mg, 200 mg, 300 mg, 400 mg, 500 mg, 1000 mg, 2000 mg, or 10000 mg for the treatment of a sleep breathing disorder. In one example, the KCNQ potassium channel opener is administered to a subject as a maximum daily dosage of less than 10000 mg, 2000 mg, 1500 mg, 1000 mg, 500 mg, 400 mg, 350 mg, 300 mg, 250 mg, 200 mg, 150 mg, 100 mg, 50 mg, 40 mg, 30 mg, 20 mg, 10 mg, or 5 mg for the treatment of a sleep breathing disorder. In one example, the KCNQ potassium channel opener is administered to a subject as a maximum daily dosage of between about 1 mg and 10000 mg, 20 mg and 2000 mg, 30 mg and 1500 mg, 40 mg and 1000 mg, 50 mg and 500 mg, 50 mg and 400 mg, or 50 mg and 300 mg for the treatment of a sleep breathing disorder. The KCNQ potassium channel opener may be administered to a subject as a single daily dose or as a multiple daily dose for the treatment of a sleep breathing disorder. In one example, the KCNQ potassium channel opener is administered to a subject as a single daily dose for the treatment of a sleep breathing disorder. In one example, the KCNQ potassium channel opener is administered to a subject as a multiple daily dose for the treatment of a sleep breathing disorder. For example, a maximum daily dose of the KCNQ potassium channel opener of 300 mg, may be administered to the subject as a 100 mg three times daily dose for the treatment of a sleep breathing disorder.

In some embodiments, the KCNQ potassium channel opener administered to the subject for the treatment of a sleep breathing disorder is Retigabine. In one example, Retigabine is administered to a subject as a maximum daily dosage of about 1 mg, 5 mg, 10 mg, 20 mg, 30 mg, 40 mg, 50 mg, 100 mg, 150 mg, 200 mg, 250 mg, 300 mg, 350 mg, 400 mg, 500 mg, 750 mg, or 1000 mg for the treatment of a sleep breathing disorder. In one example, Retigabine is administered to a subject as a maximum daily dosage of at least 1 mg, 5 mg, 10 mg, 20 mg, 30 mg, 40 mg, 50 mg, 100 mg, 150 mg, 200 mg, 250 mg, 300 mg, 350 mg, 400 mg, 500 mg, 750 mg, or 1000 mg for the treatment of a sleep breathing disorder. In one example, Retigabine is administered to a subject as a maximum daily dosage of less than 5000 mg, 2000 mg, 1500 mg, 1000 mg, 500 mg, 400 mg, 350 mg, 300 mg, 250 mg, 200 mg, 150 mg, 100 mg, 50 mg, 40 mg, 30 mg, 20 mg, 10 mg, or 5 mg for the treatment of a sleep breathing disorder. In one example, Retigabine is administered to a subject as a maximum daily dosage of between about 1 mg and 5000 mg, 20 mg and 2000 mg, 30 mg and 1500 mg, 40 mg and 1000 mg, 50 mg and 500 mg, 50 mg and 400 mg, or 50 mg and 300 mg for the treatment of a sleep breathing disorder. The Retigabine may be administered to a subject as a single daily dose or as a multiple daily dose for the treatment of a sleep breathing disorder. In one example, Retigabine is administered to a subject as a single daily dose for the treatment of a sleep breathing disorder. In one example, Retigabine is administered to a subject as a multiple daily dose for the treatment of a sleep breathing disorder. For example, a maximum daily dose of Retigabine of 300 mg, may be administered to the subject as a 100 mg three times daily dose for the treatment of a sleep breathing disorder. In one example, Retigabine is administered to a subject as a single daily dose of 400 mg.

In some embodiments, the KCNQ potassium channel opener administered to the subject for the treatment of a sleep breathing disorder is Flupirtine. In one example, Flupirtine is administered to a subject as a maximum daily dosage of about 1 mg, 5 mg, 10 mg, 20 mg, 30 mg, 40 mg, 50 mg, 100 mg, 150 mg, 200 mg, 250 mg, 300 mg, 350 mg, 400 mg, 500 mg, 750 mg, or 1000 mg for the treatment of a sleep breathing disorder. In one example, Flupirtine is administered to a subject as a maximum daily dosage of at least 1 mg, 5 mg, 10 mg, 20 mg, 30 mg, 40 mg, 50 mg, 100 mg, 150 mg, 200 mg, 250 mg, 300 mg, 350 mg, 400 mg, 500 mg, 750 mg, or 1000 mg for the treatment of a sleep breathing disorder. In one example, Flupirtine is administered to a subject as a maximum daily dosage of less than 5000 mg, 2000 mg, 1500 mg, 1000 mg, 500 mg, 400 mg, 350 mg, 300 mg, 250 mg, 200 mg, 150 mg, 100 mg, or 50 mg for the treatment of a sleep breathing disorder. In one example, Flupirtine is administered to a subject as a maximum daily dosage of between about 1 mg and 5000 mg, 20 mg and 2000 mg, 30 mg and 1500 mg, 40 mg and 1000 mg, 50 mg and 500 mg, 100 mg and 400 mg, 100 mg and 300 mg, or 100 mg and 200 mg for the treatment of a sleep breathing disorder. The Flupirtine may be administered to a subject as a single daily dose or as a multiple daily dose for the treatment of a sleep breathing disorder. In one example, Flupirtine is administered to a subject as a single daily dose for the treatment of a sleep breathing disorder. In one example, Flupirtine is administered to a subject as a multiple daily dose for the treatment of a sleep breathing disorder. For example, a maximum daily dose of Flupirtine of 200 mg, may be administered to the subject as a 100 mg twice daily dose for the treatment of a sleep breathing disorder. In one example, Flupirtine is administered to a subject as a single daily dose of 400 mg.

### Combination Therapies

In some embodiments, the KCNQ potassium channel opener is administered to the subject as a single therapy for the treatment or prevention of a sleep breathing disorder. That is, a single KCNQ potassium channel opener, for example, Retigabine, is administered to the subject for the treatment or prevention of a sleep breathing disorder. In one example, the KCNQ potassium channel opener is administered to the subject as a single therapy for the treatment or prevention of a sleep breathing disorder.

In some embodiments, the KCNQ potassium channel opener is administered to the subject as a combination therapy for the treatment or prevention of a sleep breathing disorder. That is, a combination of KCNQ potassium channel openers, for example, Retigabine and Flupirtine, is administered to the subject for the treatment or prevention of a sleep breathing disorder. In one example, a combination of KCNQ potassium channel openers is administered to the subject for the treatment or prevention of a sleep breathing disorder.

A combination of KCNQ potassium channel openers includes two, three, four, five, six, seven, eight, nine, ten, etc. different KCNQ potassium channel openers. In one example, a combination of two KCNQ potassium channel openers is administered to a subject for the treatment of a sleep breathing disorder. In one example, a combination of three KCNQ potassium channel openers is administered to a subject for the treatment of a sleep breathing disorder.

The combination of KCNQ potassium channel openers may include openers from the same subtype (e.g., two, three, four, five, six, seven, eight, nine, ten, etc., Kᵥ7.5 (KCNQ5) potassium channel openers). Alternatively, the combination of KCNQ potassium channel openers may include openers from two or more different subtypes (e.g., one, two, three, four, five, six, seven, eight, nine, ten, etc. Kᵥ7.5 (KCNQ5) potassium channel openers and one, two, three, four, five, six, seven, eight, nine, ten, etc. Kᵥ7.2 (KCNQ2) potassium channel openers). It will be understood that all possible combinations of KCNQ potassium channel openers may be prescribed in the treatment of a sleep breathing disorder. In one example, a combination of Retigabine and Flupirtine is administered to the subject for the treatment or prevention of a sleep breathing disorder.

In some embodiments, the KCNQ potassium channel opener, or combination thereof, is administered to the subject as a therapy for the treatment or prevention of a sleep breathing disorder in combination with another therapeutic compound. The administration of a KCNQ potassium channel opener in combination with another therapeutic compound may result in a synergistic effect in the treatment of a sleep breathing disorder. Such other therapeutic compounds include, for example, those compounds belonging to the class of purinergic receptor antagonists (e.g., P2X3 receptor antagonists), dopamine receptor agonists (e.g., dopamine receptor D2 agonists), alpha-2 adrenergic receptor agonists, GABA_{A} receptor agonists, H₃ receptor antagonists, and modulators of H₂S and CO mediated transduction mechanisms. In one example, the other therapeutic is a purinergic receptor antagonist. In one example, the other therapeutic is a dopamine receptor agonist. Examples of a dopamine receptor agonists include, but are not limited to, apomorphine, bromocriptine, cabergoline, carmoxirole, ciladopa, dihydrexidine, dinapsoline, doxanthrine, epicriptine, fendoldopam, lisuride, pergolide, piribedil, pramipexole, propylnorapomorphine, quinagolide, ropinirole, rotigotine, roxindole, and sumanirole. In one example, the dopamine receptor agonist is carmoxirole. In one example, the other therapeutic is an alpha-2 adrenergic receptor agonist. Examples of alpha-2 adrenergic receptor agonists include, but are not limited to, clonidine, dexmedetomidine, fadolmidine, guanfacine, guanabenz, guanoxabenz, guanethidine, xylazine, tizanidine, medetomidine, methyldopa, methylnorepinephrine, norepinephrine, amitraz, detomidine, lofexidine, and nolomirole. In one example, the alpha-2 adrenergic receptor agonist is nolomirole. In one example, the other therapeutic is a GABA_{A} receptor agonist. In one example, the other therapeutic is a H₃ receptor antagonist. In one example, the other therapeutic is a modulator of H₂S and CO mediated transduction mechanisms. In one example, the other therapeutic is a P2X3 receptor antagonist. In one example, a KCNQ potassium channel opener is administered in combination with a purinergic receptor antagonist. In one example, a KCNQ potassium channel opener is administered in combination with a dopamine receptor agonist. In one example, a KCNQ potassium channel opener is administered in combination with an alpha-2 adrenergic receptor agonist. In one example, a KCNQ potassium channel opener is administered in combination with a GABA_{A} receptor agonist. In one example, a KCNQ potassium channel opener is administered in combination with a H₃ receptor antagonist. In one example, a KCNQ potassium channel opener is administered in combination with a modulator of H₂S and CO mediated transduction mechanisms. In one example, a KCNQ potassium channel opener is administered in combination with a P2X3 receptor antagonist.

A combination of any two or more other therapeutic compounds may be administered with the KCNQ potassium channel opener. It will be understood that all possible combinations of KCNQ potassium channel openers in combination with all combinations of other therapeutic compounds may be prescribed in the treatment of a sleep breathing disorder. In one example, a combination of a KCNQ potassium channel opener and another therapeutic compound is administered to the subject for the treatment or prevention of a sleep breathing disorder. In one example, a combination of Retigabine and another therapeutic compound is administered to the subject for the treatment or prevention of a sleep breathing disorder. In one example, a combination of Retigabine, Flupirtine and another therapeutic compound is administered to the subject for the treatment or prevention of a sleep breathing disorder.

### Diagnosis

The subject may be assessed for suffering from or having a predisposition to a sleep breathing disorder prior to the administration of a KCNQ potassium channel opener. It will be appreciated that a sleep breathing disorder, particularly a sleep apnea, may be diagnosed by the combined evaluation of symptoms, risk factors and observation. However, the most accurate method for diagnosing a sleep breathing disorder is through a formal sleep study (e.g., polysomnography). Such a sleep study may evaluate any one or more of a subject's sleep state, eye movement, muscle activity, heart rate, respiratory effort, airflow, and blood oxygen levels.

Specifically, diagnosing a sleep breathing disorder may involve testing for elevated loop gain. The person skilled in the art would appreciate that the published literature contains methods for the measurement of loop gain in patients with central sleep apnea and those with obstructive sleep apnea.

Suitable methods for testing for elevated loop gain are described in Sands et al. (2011) and Terrill et al. (2011).

Another suitable method involves a simple breath-hold technique to estimate loop gain in awake, healthy volunteers and obstructive sleep apnea patients (such as those described in Messineo et al. (2018)). This method is based on the finding that maximal breath-hold duration and the subsequent hyperventilatory response correlates with gold-standard measures of loop gain, namely, ventilator responses to hypercapnic and hypoxic pulses and CPAP pressure drops. These breath-hold manoeuvres offer an alternative method for assessing the predisposition to recurrent apnea and hypopneas. This simple technique can be performed in the physician's office without special equipment, and is readily adaptable to the ambulatory setting.

In some embodiments, the subject is tested for elevated loop gain prior to or following administration of a KCNQ potassium channel opener. In one example, the subject is tested for elevated loop gain prior to administration of a KCNQ potassium channel opener. In one example, the subject is tested for elevated loop gain following administration of a KCNQ potassium channel opener. In one example, the subject is tester for elevated loop gain both prior to administration of a KCNQ potassium channel opener and following administration of a KCNQ potassium channel opener. A decrease in loop gain following the administration of a KCNQ potassium channel opener would be understood to be indicative of an improvement in sleep apnea, and therefore indicative of an effective sleep breathing disorder treatment.

It is therefore possible to assess the effectiveness of a sleep breathing disorder treatment (e.g., administration of a KCNQ potassium channel opener) in a manner comparable to its diagnosis. That is, the effectiveness of a sleep breathing disorder treatment (e.g., administration of a KCNQ potassium channel opener) may be assessed by testing for reduced loop gain. In one example, the administration of a KCNQ potassium channel opener to a subject suffering from a sleep breathing disorder results in a reduced loop gain. In one example, the administration of a KCNQ potassium channel opener to a subject suffering from a sleep breathing disorder results in an activated M current.

In one example, an improvement in sleep apnea in a subject following administration of a KCNQ potassium channel opener is detected by conducting a sleep study. The sleep study may assess at least one of the subject's sleep state, eye movement, muscle activity, heart rate, respiratory effort, airflow, blood oxygen levels, arterial PCO₂, and arterial H⁺ concentration, as would be understood by the person skilled in the art to relate to an improvement in sleep apnea in a subject. As a decrease in arterial PCO₂ may be observed in a subject suffering from a sleep breathing disorder, such as a sleep apnea, it follows that an increase in PCO₂ may be observed following an improvement in a breathing disorder in a subject, such as sleep apnea. Similarly, as a decrease in arterial H⁺ concentration may be observed in a subject suffering from a sleep breathing disorder, such as a sleep apnea, it follows that an increase in PCO₂ may be observed following an improvement in a breathing disorder in a subject, such as a sleep apnea. Accordingly, in one example, an improvement in sleep apnea in a subject following administration of a KCNQ potassium channel opener is detected by an increase in arterial PCO₂. In one example, an improvement in sleep apnea in a subject following administration of a KCNQ potassium channel opener is detected by an increase in arterial H⁺ concentration.

### Formulations

The person skilled in the art will appreciate that the KCNQ potassium channel opener may be appropriately formulated into a pharmaceutical composition for administration to the subject. The pharmaceutical compositions may be suitable for use in a variety of drug delivery systems. Suitable formulations for use in the present disclosure may be found in Remington's Pharmaceutical Sciences, Mack Publishing Company, Philadelphia, Pa., 17th ed. (1985). For a brief review of methods for drug delivery, see Langer (1990).

For preparing pharmaceutical compositions containing a KCNQ potassium channel opener, inert and pharmaceutically acceptable carriers are used. As used herein, the term “pharmaceutically acceptable carrier” includes any and all solids or solvents (such as phosphate buffered saline buffers, water, saline) dispersion media, coatings, antibacterial and antifungal agents, isotonic and absorption delaying agents, and the like, compatible with pharmaceutical administration. The pharmaceutically acceptable carriers must be ‘acceptable’ in the sense of being compatible with the other ingredients of the composition and not deleterious to the recipient thereof. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material. In powders, the carrier is generally a finely divided solid that is in a mixture with the finely divided active component. In tablets, the active ingredient (i.e., a KCNQ potassium channel opener) is mixed with the carrier having the necessary binding properties in suitable proportions and compacted in the shape and size desired.

The amount of pharmaceutically acceptable carrier will depend upon the level of the compound and any other optional ingredients that a person skilled in the art would classify as distinct from the carrier (e.g., other active agents). The formulations of the present invention may comprise, for example, from about 5% to 99.99%, or 25% to about 99.9% or from 30% to 90% by weight of the composition, of a pharmaceutically acceptable carrier. The pharmaceutically acceptable carrier can, in the absence of other adjuncts, form the balance of the composition.

Optionally, the pharmaceutical composition of the present disclosure further comprises other additional components, for example therapeutic and/or prophylactic ingredients. The present disclosure thus relates in a further aspect to pharmaceutical composition comprising the compound of the present disclosure, one or more pharmaceutically acceptable carriers together with one or more other active agents. Generally, the amount of other active agent present in the pharmaceutical composition is sufficient to provide an additional benefit either alone or in combination with the other ingredients in the composition.

It will be understood by the person skilled in the art that these optional components may be categorized by their therapeutic or aesthetic benefit or their postulated mode of action. However, it is also understood that these optional components may, in some instances, provide more than one therapeutic or aesthetic benefit or operate via more than one mode of action. Therefore, classifications herein are made for the sake of convenience and are not intended to limit the component to that particular application or applications listed. Also, when applicable, the pharmaceutically-acceptable salts of the components are useful herein.

When other active agents are present in the pharmaceutical formulation of the present invention, the dose of the compound may either be the same as or differ from that employed when the other additional components are not present. Appropriate doses will be readily appreciated by those skilled in the art.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets may contain between about 5% to about 70% by weight of the active ingredient of an angiotensin II signalling inhibitor. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a KCNQ potassium channel opener with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a KCNQ potassium channel opener) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a KCNQ potassium channel opener) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, for example from 5 to 9, or from 7 to 8.

Single or multiple administrations of the pharmaceutical compositions can be carried out with dose levels and pattern being selected by the treating practitioner. In any event, the pharmaceutical formulations should provide a quantity of an angiotensin II signalling inhibitor sufficient to effectively treat or prevent a viral infection in the patient.

When used for pharmaceutical purposes, the KCNQ potassium channel opener may be generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. (1966).

The compositions can additionally include a stabilizer, enhancer or other pharmaceutically acceptable carriers or vehicles. A pharmaceutically acceptable carrier can contain a physiologically acceptable compound that acts, for example, to stabilize the compounds. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

The formulations containing a KCNQ potassium channel opener may be delivered to any tissue or organ using any delivery method known to the ordinarily skilled artisan. They may be formulated for subcutaneous, intramuscular, intravenous, intraperitoneal, or intratumor injection, or for oral ingestion or for topical application. Effective dosage of the formulations will vary depending on many different factors, including means of administration, target site, physiological state of the patient, and other medicines administered. Thus, treatment dosages will need to be titrated to optimize safety and efficacy. In determining the effective amount of a compound to be administered, the physician should evaluate the particular compound being used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector. Doses may generally range between about 0.01 and about 100 μg per kilogram of body weight, for example between about 0.1 and about 50 μg per kg of body weight.

In some embodiments, the pharmaceutical composition comprises a therapeutically effective amount of a KCNQ potassium channel opener. The content of the KCNQ potassium channel opener in the pharmaceutical composition is, for example, from about 0.1% to about 100% w/w of the pharmaceutical composition. In one example, the pharmaceutical composition comprises a therapeutically effective amount of a KCNQ potassium channel opener. In one example, the pharmaceutical composition comprises a therapeutically effective amount of Retigabine. In one example, the pharmaceutical composition comprises a therapeutically effective amount of Flupirtine. In one example, the pharmaceutical composition comprises a therapeutically effective amount of a KCNQ potassium channel opener and another therapeutic compound.

The present disclosure provides pharmaceutical formulations or compositions, both for veterinary and for human medical use, which comprise one or more KCNQ potassium channel openers, which may or may not be in combination with one or more other therapeutic compounds, or any embodiments thereof as described herein or any pharmaceutically acceptable salts thereof, with one or more pharmaceutically acceptable carriers and/or excipients, and optionally any other therapeutic ingredients, stabilisers, or the like.

The carrier(s) or excipients must be pharmaceutically acceptable in the sense of being compatible with the other ingredients, such as sugars, hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

The present disclosure will now be described further with reference to the following examples, which are illustrative only and non-limiting. The examples refer to the figures.

### EXAMPLES

### Example 1: Effect of the KCNQ Channel Openers Retigabine and Flupirtine on Periodic Breathing in an Animal Model of Elevated Loop Gain Associated with Increased Chemosensitivity

Aim

To determine the effects of the KCNQ channel openers Retigabine and Flupirtine on loop gain and periodic breathing in the anaesthetised, hyperventilated Domperidone-treated lamb.

Approach

Edwards et al. (2008) demonstrated that administration of the dopamine D2-receptor antagonist Domperidone to young lambs increases the loop gain of the respiratory control system and increases the likelihood that an epoch of disordered breathing will occur following a transient perturbation of the breathing pattern. In particular, Edwards et al. (2008) showed that the increase in loop gain induced by domperidone is due predominately to an increase in the controller gain for oxygen, reflecting increased carotid body chemosensitivity. This animal model provides a robust preparation to test the effects of candidate drugs for the treatment of disordered breathing associated with increased peripheral chemosensitivity and elevated loop gain.

Method

Animal Model

Young lambs (Merino/Border-Leicester cross, 14-18 kg) were instrumented surgically and maintained through the experimental protocol according to the method described by Edwards et al. (2008). Briefly, a non-occlusive catheter was placed in the left jugular vein for the induction of anaesthesia using a loading dose of ketamine hydrochloride (5 mg/kg) followed by alpha-chloralose (80 mg/kg as a starting bolus, then as a continuous infusion at 20 mg/kg/hr. Supplemental doses of alpha-chloralose were administered as needed to maintain adequate anaesthesia. A glucose-saline solution was infused through the same catheter to maintain fluid levels (5% glucose in 0.9% saline at 4 ml/kg/hr). A tracheal tube was inserted and connected to a dual intermittent mechanical ventilator (IMV)/high frequency oscillatory ventilator (Humming2; BMO-20H) used in the IMV mode. Use of a 4-way Hans Rudolf valve enabled the lamb to be ventilated artificially or to breathe spontaneously from a circuit through which a flow of gas was maintained at 45 L/min, or from the room air. Measurements of respiratory flow, tidal volume and end-expired gases were made on the tracheal line. Respiratory flow was measured using a Hans Rudolph (3500A) pneumotachograph that was linear up to 35 l/min, in conjunction with a Gaeltec 8T-2 differential pressure transducer connected to a carrier amplifier (Hewlett Packard 8805B) and thence to an HP-8815A electronic integrator to determine tidal volume. Non-occlusive catheters were placed in the right jugular vein and a carotid artery for the intravenous administration of drugs, the sampling of venous and arterial blood gases and the monitoring of blood pressure. Arterial blood pressure was measured using a Micron MP-15 blood pressure transducer connected to a Hewlett-Packard 8805B carrier amplifier. A rectal probe was used to monitor body temperature, which was maintained within the range of 40.0±1.0° C. using an overhead radiant heater. Arterial oxygen saturation was measured continuously using a Nellcor pulse oximeter (Model N-200E) with the probe placed in the trans-mandibular position in such a way that the optical path included the soft tissues of the jaw but excluded the tongue. All physiological signals were digitized at 400 Hz and captured and displayed on a computer using Powerlab hardware and Chart 5 software (ADInstruments, Sydney Australia).

Drugs

Domperidone (0.5 mg/kg in 30 ml 0.1% lactic acid), Flupirtine (as Flupirtine maleate, 5 mg/kg in 30 ml distilled water) and Retigabine (5 mg/kg in 30 ml distilled water) were each administered over a period of 5 minutes via the jugular vein catheter.

Protocol

Lambs breathed room air (atmospheric) throughout the experimental protocol. Domperidone was administered at the commencement of the experimental protocol. Previous research has demonstrated that the blocking effect of a 0.5 mg/kg dose of domperidone to a lamb is maintained for at least three hours (see also Kressin et al. 1986). Epochs of periodic breathing were induced by switching the tracheal tube to the ventilator circuit and hyperventilating the lamb with air for at least 5 minutes to reduce end tidal CO₂ to a level that produced apnea when the ventilator was switched off. The level of hyperventilation was adjusted so as to generate a post-hyperventilation apnea duration that resulted in arterial oxygen saturation falling to approximately 50-60% before the animal began to breathe spontaneously. Typically, an extended epoch of periodic breathing followed the post-hyperventilation apnea. Once the ventilation parameters for achieving a post-hyperventilation apnea as described above were established for a particular animal, the baseline ventilatory response to hyperventilation was determined by performing two standardised hyperventilation procedures 10-15 minutes apart. The test drug (Flupirtine or Retigabine) was then administered and, approximately 1 hour later, another two hyperventilation procedures were performed 10-15 minutes apart.

Analysis

Minute ventilation was calculated breath-by-breath using the breath duration determined from the respiratory flow trace and the tidal volume signal from the integrator. Periodic breathing was defined as two or more sequential apneas of >3 seconds duration separated by a brief period of ventilation. The duty ratio method of Sands et al. (2011) was used to calculate loop gain during epochs of periodic breathing. The method entails measurement of the duty ratio for cycles of periodic breathing, where duty ratio (DR) is defined as the duration of the ventilatory phase of a cycle divided by the duration of the combined ventilatory and apneic phases of the cycle. A value of loop gain (LG) was calculated for each cycle of ventilation and apnea that occurred during the epoch using the formula LG=2π/(2πDR−sin(2πDR)). The average value for the entire epoch was then calculated.

Results

Hyperventilation of Domperidone treated lambs, 30 minutes or more after the administration of domperidone, reliably elicited epochs of periodic breathing. These epochs typically lasted 2-5 minutes, although in several instances long epochs lasting up to 10 minutes were elicited, during which there were as many as 140 successive cycles of ventilation and apnea.

Administration of either Retigabine or Flupirtine to the Domperidone-treated lamb significantly reduced the number of cycles in epochs of periodic breathing that occurred following hyperventilation before continuous breathing was re-established. Both epoch duration and the average value of loop gain across the epoch were reduced by Retigabine and Flupirtine.

Conclusion

The results indicate that the KCNQ channel openers Flupirtine and Retigabine each act to reduce loop gain and the duration of epochs of periodic breathing.

### Example 2: Effect of a KCNQ Channel Opener on Peripheral Chemoreceptor Sensitivity, Loop Gain and Sleep Disordered Breathing in Patients with Obstructive Sleep Apnea

Overview

The purpose of this study is to determine whether the administration of a KCNQ channel opener (in particular, a KCNQ2/3 opener) to an individual suffering from OSA associated with elevated LG is able to reduce the sensitivity of the peripheral chemoreceptors in that individual and improve sleep quality by reducing extent of periodic breathing during sleep. It is contemplated that the study be repeated using different KCNQ opener drugs from different chemical classes and with differing abilities to penetrate the blood brain barrier, in order to determine whether or not it may be preferable to use a KCNQ opener with relatively poor brain penetration to treat sleep disordered breathing in patients who suffer from sleep disordered breathing associated with elevated LG.

Details of Study

Aim

To determine the effect of a KCNQ channel opener (DRUG) on chemoreceptor sensitivity, loop gain (LG) and sleep disordered breathing (SDB) in individuals with OSA.

The study may be conducted using any orally-available drug that is effective at opening KCNQ2/3 potassium channels. Preferably, the study is conducted using Retigabine as the DRUG. Alternatively, the study may be conducted using Flupirtine or Benzbromarone as the DRUG, or other KCNQ channel opener for which appropriate preclinical and clinical safety studies have been completed to the satisfaction of the appropriate regulatory authority and/or institutional review board (human research ethics committee).

Hypothesis

It is hypothesised that, in patients with OSA, the administration of DRUG before sleep will reduce both LG and the severity of SDB, as measured by the apnea-hypopnea index (AHI) and other measures of sleep and breathing dysfunction.

Additionally, it is hypothesised that the ability of DRUG to reduce LG and mitigate SDB in individuals with OSA will be associated with a DRUG-associated reduction in peripheral chemoreceptor sensitivity, as measured while awake.

Methods

Study Drug (DRUG):
Preferably, Retigabine (100 mg, 150 mg or 200 mg)
   Alternatively, Flupirtine, (100 mg or 200 mg) or Benzbromarone (50 mg, 100 mg or 200 mg)

Subjects:
10 subjects (21-65 years old) with a history of moderate-to-severe OSA, as diagnosed by a qualified physician on the basis of clinical polysomnography (PSG)
   Willing to refrain from using CPAP or any other form of ventilatory assistance device for at least 7 days prior to the first dose of the DRUG (Study Night 1) and throughout the study.

Inclusion Criteria:
AHI>10 events per hour during non-rapid eye movement (NREM) sleep, as measured in a recent overnight sleep study (clinical PSG)

Exclusion Criteria:
Medical and concurrent medication exclusions in accordance with the most recently published Summary of Product Characteristics (SPC) or Package Insert (PI) for the DRUG being evaluated (retigabine, flupirtine, benzbromarone) or, for any investigative new drug, any relevant exclusions arising from pre-clinical and clinical safety studies conducted to date
History of CSA or mixed sleep apnea
Concurrent or recent (within the past month) use of any medication known to influence sleep, arousal, circadian rhythm, breathing or muscle function
Pregnancy
Occupation or life situation that may be put at risk by participation in the study
History of shift work or rotating shifts in the month prior to the first dose of the DRUG (Study Night 1)

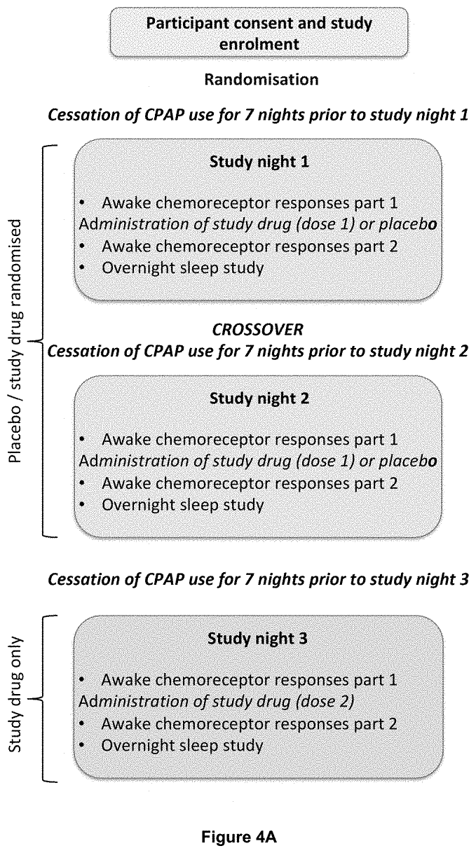

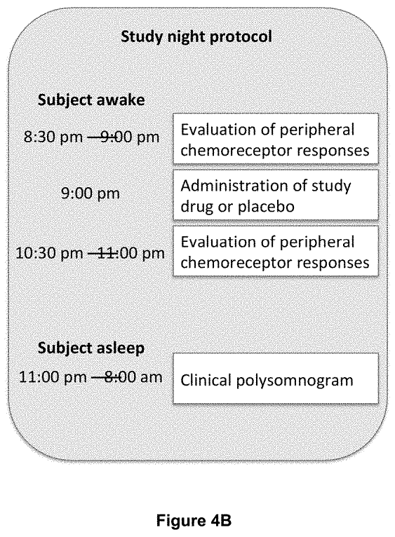

Study Design (FIGS. 4A and 4B)
Randomised, placebo-controlled double-blinded cross-over study (Study Nights 1 and 2) with optional open-label dose elevation extension (Study Night 3).

Randomisation
Study Night 1
   Evaluation of chemoreceptor sensitivity in the awake subject prior to administration of DRUG or placebo, using the transient hypoxia and hypercapnia method described by Pfoh et al. (2016).
   Administration of DRUG at the starting dose approved by the relevant IRB (Retigabine 100 mg, Flupirtine 100 mg, Benzbromarone 50 mg or 100 mg), or placebo
   Evaluation of chemoreceptor sensitivity in the awake subject prior following the administration of DRUG or PLACEBO, using the method of Pfoh et al. (2016)
   Overnight sleep study with clinical PSG
Crossover
Study Night 2 (one week following Study Night 1)
   Evaluation of chemoreceptor sensitivity in the awake subject prior to administration of DRUG or PLACEBO, using the method of Pfoh et al. (2016)
   Administration of DRUG at the starting dose approved by the relevant IRB (Retigabine 100 mg, Flupirtine 100 mg, Benzbromarone 50 mg or 100 mg), or PLACEBO
   Evaluation of chemoreceptor sensitivity in the awake subject prior following the administration of DRUG or PLACEBO, using the method of Pfoh et al. (2016)
   Overnight sleep study with clinical PSG
Election by subject to proceed to optional Study Night 3 if subject did not experience any adverse events on Study Night 1 or Study Night 2
Study Night 3 (one week following Study Night 2)
   Evaluation of chemoreceptor sensitivity in the awake subject prior to administration of an elevated dose DRUG, using the method of Pfoh et al. (2016)
   Administration of DRUG at the elevated dose approved by the relevant IRB (Retigabine 150 or 200 mg, Flupirtine 200 mg, Benzbromarone 100 mg, 150 mg or 200 mg)
   Evaluation of chemoreceptor sensitivity in the awake subject prior following the administration of DRUG at the elevated dose, using the method of Pfoh et al. (2016)
   Overnight sleep study with clinical PSG

Analysis
Primary outcome measures
   Difference in the change in chemoreceptor sensitivity in the awake subject before and after the administration of DRUG or PLACEBO (Study Night 1 vs Study Night 2)
   Difference in Apnea/Hypopnea Index (AHI) during non-REM sleep between DRUG and PLACEBO (Study Night 1 vs Study Night 2)
Secondary outcome measures
   Difference in average values of Loop Gain (LG) during episodes of periodic breathing during sleep (measured using the method of Terrill et al. (2015) in the presence or absence of DRUG (Study Night 1 vs Study Night 2)
   Difference in sleep architecture measures (% time in different sleep states) in the presence or absence of DRUG (Study Night 1 vs Study Night 2)
   Effect of an elevated dose of DRUG on differences in chemoreceptor sensitivity, AHI, average LG, DT and sleep architecture (Study Night 3 vs Study Night 1 and Study Night 2)

Results

Administration of a KCNQ channel opener to a subject with OSA that is characterised by an elevated LG was associated with a reduction in peripheral chemoreceptor sensitivity when measured while awake, together with reductions in AHI and in average LG during periodic breathing while sleeping.

Conclusion

The results indicate that OSA patients whose condition is associated with an elevated loop gain can be treated effectively by administering a KCNQ channel opener such as Retigabine, Flupirtine or Benzbromarone prior to sleep. The likely effectiveness of such therapy for a particular patient can be predicted by the reduction in peripheral chemoreceptor sensitivity that is induced in the patient while awake following administration of the KCNQ opener.

### Example 3: Effect of a KCNQ Channel Opener on Peripheral Chemoreceptor Sensitivity, Loop Gain and Sleep Disordered Breathing in Patients with Central Sleep Apnea

Overview

The approach set out above in Example 2 to evaluate the ability of a KCNQ channel opener to reduce LG and mitigate the symptoms of sleep disordered breathing in OSA patients can be applied with minor modification to study the effects of a KCNQ channel opener on peripheral chemoreceptor sensitivity, LG and sleep disordered breathing in patients with CSA

Study Design

The basic design of the study is essentially the same as set out above in Example 2 and illustrated in FIGS. 4A and 4B, with the following modifications from the respective details of Example 2:

Subjects:
10 subjects (21-65 years old) with a history of moderate-to-severe CSA, as diagnosed by a qualified physician on the basis of clinical polysomnography (PSG)

Exclusion Criteria:
History of OSA or mixed sleep apnea

Secondary Outcome Measures:
Difference in average values of Loop Gain (LG) during episodes of periodic breathing, measured using the method of Sands et al. (2011), in the presence or absence of DRUG (Study Night 1 vs Study Night 2)

Results

Administration of a KCNQ channel opener to a subject with CSA is associated with a reduction in peripheral chemoreceptor sensitivity when measured while awake, together with reductions in AHI and in average LG during periodic breathing while sleeping.

Conclusion

The results indicate that CSA patients can be treated effectively by administering a KCNQ channel opener such as Retigabine, Flupirtine or Benzbromarone prior to sleep. The likely effectiveness of such therapy for a particular patient can be predicted by the reduction in peripheral chemoreceptor sensitivity that is induced in the patient while awake following administration of the KCNQ opener.

### Example 4: Dose-Related and Time-Related Effects of the Administration of a KCNQ Channel Opener on Peripheral Chemoreceptor Sensitivity, Loop Gain and Sleep Disordered Breathing in Patients with Obstructive Sleep Apnea

Overview

The purpose of this study is to determine the effects of dose and duration of treatment with a KCNQ channel opener (in particular, a KCNQ2/3 channel opener) administered to an individual suffering from OSA. The study will identify a dose or range of doses with prima facie good tolerability and efficacy characteristics that would be appropriate to be explored more thoroughly in larger, more extended studies of efficacy and tolerability and will also determine the extent to which. Additionally, the study will investigate the possibility that the extended administration of a KCNQ channel opener might, over time, reduce the intrinsic chemoreceptor sensitivity and LG, and thus form the basis of a disease modifying strategy for OSA.

Details of Study

Aim

To determine the effect of dose and duration of treatment with a KCNQ channel opener (DRUG) on chemoreceptor sensitivity, loop gain (LG) and sleep disordered breathing (SDB) in individuals with OSA.

The study can be conducted using any orally-available drug that is effective at opening KCNQ2/3 potassium channels. Preferably, the study is conducted using Retigabine as the DRUG. Alternatively, the study can be conducted using Flupirtine or Benzbromarone as the DRUG, or other KCNQ channel opener for which appropriate preclinical and clinical safety studies have been completed to the satisfaction of the appropriate regulatory authority and/or Institutional review board (human research ethics committee).

Hypothesis

It is hypothesised that, in patients with OSA, the administration of DRUG before sleep will reduce both LG and the severity of SDB, as measured by the apnea-hypopnea index (AHI) and other measures of sleep and breathing dysfunction, in a dose-dependent manner and in a time-dependent manner (ie. number of weeks treatment with DRUG).

Additionally, it is hypothesised that the intrinsic chemoreceptor sensitivity of patients with OSA will be reduced by the repeated nightly administration of DRUG over a period of several weeks.

Methods

Study Drug (DRUG):
Preferably, Retigabine (50 mg, 100 mg, 150 mg and 200 mg)
   Alternatively, Flupirtine, (100 mg or 200 mg) or Benzbromarone (50 mg, 100 mg or 200 mg)

Subjects:
20 subjects (21-65 years old) with a history of moderate-to-severe OSA, as diagnosed by a qualified physician on the basis of clinical polysomnography (PSG)
   Willing to refrain from using CPAP or any other form of ventilatory assistance device for at least 7 days prior to the first dose of the DRUG (Study Night 1) and throughout the study (completion of Study Night 2)

Inclusion Criteria:
AHI>10 events per hour during non-rapid eye movement (NREM), as measured in a recent overnight sleep study (clinical PSG)

Exclusion Criteria:
Medical and concurrent medication exclusions in accordance with the most recently published Summary of Product Characteristics (SPC) or Package Insert (PI) for the DRUG being evaluated (Retigabine, Flupirtine, Benzbromarone) or, for any investigative new drug, any relevant exclusions arising from pre-clinical and clinical safety studies conducted to date
History of CSA or mixed sleep apnea
Concurrent or recent (within the past month) use of any medication known to influence sleep, arousal, circadian rhythm, breathing or muscle function
Pregnancy
Occupation or life situation that may be put at risk by participation in the study
History of shift work or rotating shifts in the month prior to the first dose of the DRUG (Study Night 1)

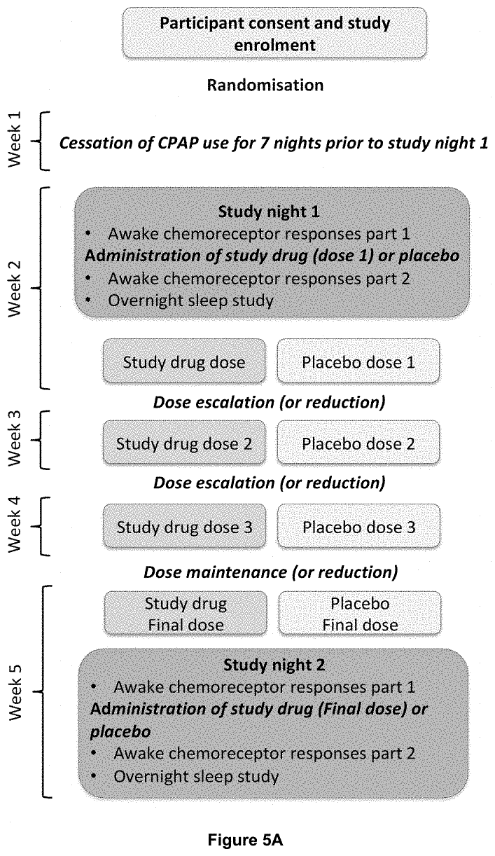

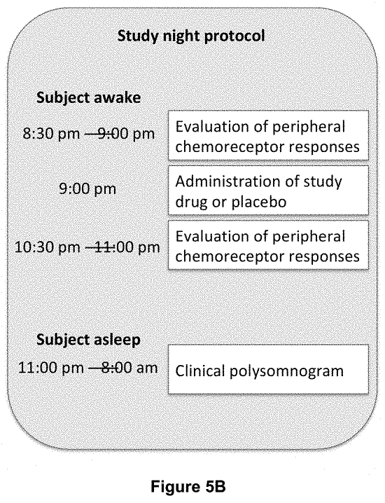

Study Design (FIGS. 5A and 5B)
Randomised, placebo-controlled double-blinded dose-escalation study
Randomisation (10 subjects to each of DRUG and Placebo arms)
Week 1 Day 1 (Study Night 1)
   Evaluation of chemoreceptor sensitivity in the awake subject prior to administration of the investigational product (IP) (DRUG or placebo), using the transient hypoxia and hypercapnia method described by Pfoh et al. (2016).
   Administration of IP at the starting dose approved by the relevant IRB (Retigabine 100 mg, Flupirtine 100 mg, Benzbromarone 50 mg or 100 mg), or placebo
   Evaluation of chemoreceptor sensitivity in the awake subject prior following the administration of IP, using the method of Pfoh et al. (2016)
   Overnight sleep study with clinical PSG
Week 1 Days 2-7
   Self administration of IP each evening prior to sleep
   Completion of questionnaire each morning
Week 2 Day 1
   Telephone interview with study coordinator. Decision on whether dose of IP for Week 2 is to be elevated (no adverse effects in Week 1 attributable to IP) or reduced (adverse effects in Week 1 that may reasonably be attributed to IP)
Week 2 Days 1-7
   Self administration of IP at adjusted dose each evening prior to sleep
   Completion of questionnaire each morning
Week 3 Day 1
   Telephone interview with study coordinator. Decision on whether dose of IP for Week 3 is to be elevated (no adverse effects in Week 2 attributable to IP) or reduced (due to the occurrence of any adverse effects in Week 2 that may reasonably be attributed to IP)
Week 3 Days 1-7
   Self administration of IP at adjusted dose each evening prior to sleep
   Completion of questionnaire each morning
Week 4 Day 1
   Telephone interview with study coordinator. Decision on whether dose of DRUG for Week 4 is to be elevated (no adverse effects in Week 3 attributable to DRUG) or reduced (due to the occurrence of any adverse effects in Week 3 that may reasonably be attributed to DRUG)
Week 4 Days 1-7
   Self administration of IP at adjusted dose each evening prior to sleep
   Completion of questionnaire each morning
Week 5 Day 1
   Telephone interview with study coordinator. Decision on whether dose of IP for Week 5 is to be maintained (no adverse effects in Week 4 attributable to IP) or reduced (due to the occurrence of any adverse effects in Week 4 that may reasonably be attributed to IP)
Week 4 Days 1-6
   Self administration of IP at adjusted dose each evening prior to sleep
   Completion of questionnaire each morning
Week 5 day 7 Study Night 2
   Evaluation of chemoreceptor sensitivity in the awake subject prior to administration of IP, using the method of Pfoh et al. (2016).
   Administration of IP at the dose used on Days 1-6 of Week 5
   Evaluation of chemoreceptor sensitivity in the awake subject prior following the administration of IP, using the method of Pfoh et al. (2016).
   Overnight sleep study with clinical PSG

DRUG Escalation and De-Escalation Regimen:

| Drug | Dose | Escalate to | Reduce to |
|---|---|---|---|
| Retigabine | 50 mg | 100 mg | 0 mg |
| | 100 mg | 150 mg | 50 mg |
| | 150 mg | 200 mg | 100 mg |
| Flupirtine | 100 mg | 150 mg | 0 mg |
| | 150 mg | 200 mg | 100 mg |
| Benzbromarone | 50 mg | 100 mg | 0 mg |
| | 100 mg | 150 mg | 50 mg |
| | 150 mg | 200 mg | 100 mg |

Analysis
Primary outcome measures:
   Effect of DRUG on the difference between Apnea/Hypopnea Index (AHI) during non-REM sleep measured on Study Night 1 and Study Night 2
   Effect of DRUG on the difference between awake chemoreceptor sensitivity measured on Study Night 1 and Study Night 2 prior to administration of IP
Secondary outcome measures:
   Effect of DRUG on the difference between in average values of Loop Gain (LG) during episodes of periodic breathing measured on Study Night 1 and Study Night 2 using the method of Terrill et al. (2015).
   Effect of DRUG on differences between sleep architecture measures (% time in different sleep states measured on Study Night 1 and Study Night 2
   Effect of the final (Week 5) dose of DRUG on the difference between Apnea/Hypopnea Index (AHI) during non-REM sleep measured on Study Night 1 and Study Night 2

Results

Administration of a KCNQ channel opener to a subject with OSA is associated with a reduction in peripheral chemoreceptor sensitivity when measured while awake, together with reductions in AHI and in average LG during periodic breathing while sleeping.

Conclusion

The results indicate that OSA patients can be treated effectively by administering a KCNQ channel opener such as Retigabine, Flupirtine or Benzbromarone prior to sleep. The likely effectiveness of such therapy for a particular patient can be predicted by the reduction in peripheral chemoreceptor sensitivity that is induced in the patient while awake following administration of the KCNQ opener.

### Example 5: Dose-Related and Time-Related Effects of the Administration of a KCNQ Channel Opener on Peripheral Chemoreceptor Sensitivity, Loop Gain and Sleep Disordered Breathing in Patients with Central Sleep Apnea

Overview

The approach set out above in Example 4 to evaluate the ability of a KCNQ channel opener to reduce LG and mitigate the symptoms of sleep disordered breathing in OSA patients may be applied with minor modification to study the effects of a KCNQ channel opener on peripheral chemoreceptor sensitivity, LG and sleep disordered breathing in patients with CSA.

Study Design

The basic design of the study is essentially the same as set out above in Example 4 and illustrated in FIGS. 5A and 4B, with the following modifications from the respective details of Example 4:

Subjects:
20 subjects (21-65 years old) with a history of moderate-to-severe CSA, as diagnosed by a qualified physician on the basis of clinical polysomnography (PSG)
Willing to refrain from using CPAP or any other form of ventilatory assistance device for at least 7 days prior to the first dose of the DRUG (Study Night 1) and throughout the study (completion of Study Night 2)

Exclusion Criteria:
History of OSA or mixed sleep apnea

Secondary Outcome Measures:
Effect of DRUG on the difference between in average values of Loop Gain (LG) during episodes of periodic breathing measured on Study Night 1 and Study Night 2 using the method of Sands et al. (2011).

Results

Administration of a KCNQ channel opener to a subject with CSA is associated with a reduction in peripheral chemoreceptor sensitivity when measured while awake, together with reductions in AHI and in average LG during periodic breathing while sleeping.

Conclusion

The results indicate that CSA patients can be treated effectively by administering a KCNQ channel opener such as Retigabine, Flupirtine or Benzbromarone prior to sleep. The likely effectiveness of such therapy for a particular patient can be predicted by the reduction in peripheral chemoreceptor sensitivity that is induced in the patient while awake following administration of the KCNQ opener.

### Example 6: Identification of Therapeutic Agents that Act Synergistically with KCNQ Channel Openers to Reduce Peripheral Chemoreceptor Sensitivity

Details of Study

Aim

To identify classes of drugs which reduce the responsiveness of carotid body chemoreceptors to a hypoxic stimulus when KCNQ channel function is blocked pharmacologically.

Hypothesis

It is hypothesised that the sensitivity of carotid body chemoreceptors may be able to be reduced by the administration of drugs whose mechanism of action does not depend of the function of KCNQ channels. Such drugs would be leading candidates for administration in combination with KCNQ channel openers to treat sleep disordered breathing.

Methods

In vitro preparation of the rat carotid body for recording afferent neural activity from the carotid sinus nerve in response to hypoxic stimuli. The method described by Allen (1998) is used to prepare an isolated rat carotid body in a temperature-controlled organ bath so that that the activity of afferent neurons can be recorded from the cut end of the carotid sinus nerve using a polished glass suction electrode. The preparation is superfused continuously with physiological saline (Tyrode's solution) containing 10-30 μM XE991 (10,10-bis(4-pyridinylmethyl)-9(10H)-anthracenone) to block KCNQ channels. The superfusate may be switched swiftly between a saline solution that has been equilibrated with a normoxic gas mixture (21% 02, 5% CO₂, balance N₂) and a solution equilibrated with a hypoxic mixture (5% 02, 5% CO₂, balance N₂). The test drug is added to the normoxic and hypoxic superfusates in increasing amounts during the course of an experiment, in order to generate standard concentrations of drug for the subsequent determination of dose response characteristics.

Protocol

Neural signals recorded with the electrode are amplified (1,000×-10,000×) and filtered (10 Hz-3 KhZ) and then digitised (10 kHz), captured and displayed using an appropriate computer-based recording system (e.g. PowerLab, ADInstruments). The chemoreceptor response to a hypoxic stimulus is quantified in terms of the change in the integrated signal from the suction electrode measured over an interval of 30 seconds, one minute prior to the onset of the hypoxic stimulus and one minute after the replacement of the normoxic solution with a hypoxic solution.

At the start of each experiment, the response of the carotid body chemoreceptors to a hypoxic stimulus in the absence of any test drug is measured by switching the superfusate from normoxic to hypoxic for 2 minutes. The superfusate is then switched back to normoxic for 10-15 minutes and then the hypoxic test repeated. Thereafter, the test drug is introduced to both nomoxic and hypoxic superfusates in stepped amounts, giving a starting concentration of 10 nM and incrementing in semi-log steps to a final concentration of 100 μM. The carotid body chemoreceptor response to a hypoxic stimulus is measured twice at each concentration of test drug, with intervals (10-15 minutes) of normoxic superfusion separating each hypoxic test.

Examples of the drugs to be tested in this study are listed in Table 1.

Outcomes

The data obtained are used to generate a dose-response curve for each test drug.

The drugs, or classes of drugs, exhibiting the most favourable dose-response characteristics (greatest suppression of the chemoreceptor response to a hypoxic stimulus at the lowest concentration of applied drug) represent preferred drug candidates to be administered in combination with a KCNQ channel opener to treat sleep disordered breathing associated with elevated loop gain. The efficacy of these combinations in reducing loop gain and treating sleep disordered breathing is evaluated further in animal and human models, as described above in Examples 1, 2, 3, 4 and 5.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Allen A M. (1998) Angiotensin AT₁ receptor-mediated excitation of rat carotid body chemoreceptor afferent activity. J. Physiol. 510: 773-781
Dempsey J A, Veasey S C, Morgan B J, O'Donnell C P. (2010) Pathophysiology of sleep apnea. Physiol. Rev. 90: 47-112.
Edwards B A, Sands S A, Skuza E M, Stockx E M, Brodecky V, Wilkinson M H, Berger P J. (2008) Increased peripheral chemosensitivity via dopaminergic manipulation promotes respiratory instability in lambs. Respir. Physiol. Neurobiol. 164: 419-428
Francis D S, Wilson K, Davies L C, Coats A J, Piepoli M (2000) Quantitative general theory for periodic breathing in chronic heart failure and its clinical implications. Circulation 102: 2214-2221
Good N E, Winget G D, Winter W, Connolly T N, Izawa S, Singh R M M. (1966) Hydrogen ion buffers for biological research. Biochemistry 5: 467-477
Khoo M C K, Kronauer R E, Strohl K P, Slutsky A S. (1982) Factors inducing periodic breathing in humans: a general model. J. Appl. Physiol. 53:644-659
Kressin N A, Nielsen A M, Laravuso R, Bisgard G E. (1986) Domperidone-induced potentiation of ventilator responses in awake goats. Respir. Physiol. 65: 169-180 Langer R. (1990) New methods of drug delivery. Science 249: 1527-1533
Messineo L, Taranto-Montemurro L, Azabarzin A, Marques M D O, Calianese N, White D P, Wellman A, Sands S A. (2018) Breath-holding as a means to estimate loop gain contribution to obstructive sleep apnoea. J. Physiol. 596: 4043-4056
Pfoh J R, Tymko M M, Abrosimova M, Boulet L M, Foster G E, Bain A R, Ainslie P N, Steinback C D, Bruce C D, Day T A. (2016) Comparing and characterizing transient and steady-state tests of the peripheral chemoreflex in humans. Exp. Physiol. 101: 432-447.
Sands Sa, Edwards B A, Kee K, Turton A, Skuza E M, Roebuck T, O'Driscoll D M, Hamilton G S, Naughton M T, Berger P J. (2011) Loop gain as a means to predict a positive airway pressure suppression of Cheyne-Stokes respiration in patients with heart failure. Am. J. Respir. Crit. Care. Med. 184: 1067-1075
Terrill P I, Edwards B A, Nemati S, Butler J P, Owens R L, Eckert D J, White D P, Malhotra A, Wellman A, Sands S A. (2015) Eur. Respir. J. 45: 408-418

1-29. (canceled)

30. A method of treating or preventing a sleep breathing disorder associated with elevated loop gain in a subject, the method comprising administering an effective amount of a KCNQ potassium channel opener selected from a compound of Formula I or II: 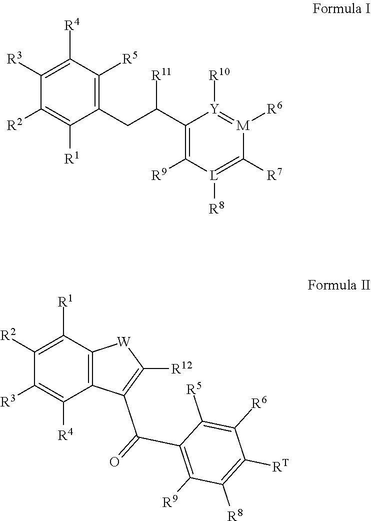
wherein
n is 1 or 2;
when n is 1, Q is CR¹⁴R¹⁵ or C(O); and when n is 2, Q are each independently CR¹⁴R¹⁵ or C(O);
Y, M and L are each independently C or N;
when Y is N, R¹⁰ is not present; when M is N, R⁶ is not present; and when L is N, R⁸ is not present;
W is O, S or NH;
R¹, R², R³, R⁴, R⁵, R⁶, R⁷, R⁸, R⁹, R¹⁰, and R¹² are each independently selected from hydrogen, halo, CF₃, CN, NO₂, OH, SH, NH₂, S(O)OH, C(O)H, C(O)₂H, OC(O)R¹⁴, C(O)R¹⁴, C(O)NR¹⁴R¹⁵, C(O)OR¹⁴, OR¹⁴, NHC(O)OR¹⁴, OS(O)₂R¹⁴, S(O)₂NR¹⁴R¹⁵, NR¹⁴R¹⁵, SR¹⁴, C₁₋₂₀alkyl-C(O)OR¹⁴, C₁₋₂₀alkyl, C₂₋₂₀alkenyl, C₂₋₂₀ alkynyl, monocyclic or polycyclic carbocyclic, and monocyclic or polycyclic heterocyclic;
wherein R¹⁴ and R¹⁵ are each independently selected from H and C₁₋₁₀alkyl, wherein the C₁₋₁₀alkyl is optionally interrupted with one or more heteroatoms independently selected from O, N and S; and wherein the C₁₋₁₀alkyl is optionally substituted with one or more substituents independently selected from the group consisting of hydrogen, halo, CF₃, CN, NO₂, OH, SH, NH₂, S(O)OH, C(O)H and C(O)₂H; and
wherein the C₁₋₂₀ alkyl, C₂₋₂₀ alkenyl, and C₂₋₂₀ alkynyl are each optionally interrupted with one or more heteroatoms independently selected from O, N and S; and wherein the C₁₋₂₀alkyl, C₂₋₂₀alkenyl, C₂₋₂₀alkynyl, monocyclic or polycyclic carbocyclic, and monocyclic or polycyclic heterocyclic are each optionally substituted with one or more substituents independently selected from the group consisting of halo, CF₃, CN, NO₂, OH, SH, NH₂, S(O)OH, C(O)H and C(O)₂H; and
R¹¹ is selected from the group consisting of hydrogen and C₁₋₁₀alkyl.

31. The method according to claim 30, wherein the KCNQ potassium channel opener is a KCNQ2-5 channel opener.

32. The method according to claim 31, wherein the peripheral plasma concentration of the KCNQ potassium channel opener is at least about 1.5 times greater than the central plasma concentration of the KCNQ potassium channel opener.

33. The method according to claim 30, wherein the KCNQ potassium channel opener is selected from the group consisting of Retigabine, Flupirtine, SF0034, RL648_81, Benzbromarone, ICA-169673, and Compound 40.

34. The method according to claim 30, wherein the sleep breathing disorder is selected from the group consisting of non-obstructive, or central (CSA), sleep apnea, obstructive sleep apnea (OSA), and mixed sleep apnea.

35. The method according to claim 34, wherein the central sleep apnea (CSA) is Cheyne-Stokes respiration (CSR).

36. The method according to claim 30, wherein the subject is tested for elevated loop gain prior to or following administration of the KCNQ potassium channel opener.

37. The method according to claim 30, wherein the KCNQ potassium channel opener is administered in combination with an additional therapeutic agent selected from the group consisting of purinergic receptor antagonists, dopamine receptor agonists, alpha-2 adrenergic receptor agonists, GABA_{A} receptor agonists, H₃ antihistamines, and modulators of H₂S and CO mediated transduction mechanisms.

38. The method according to claim 37, wherein the additional therapeutic agent is selected from the group consisting of a P2X3 receptor antagonist, a dopamine receptor agonist, carmoxirole, alpha-2 adrenergic receptor agonist, and nolomirole.

39. The method according to claim 30, wherein the KCNQ potassium channel opener is administered to the subject orally.

40. The method according to claim 30, wherein the KCNQ potassium channel opener is administered as a once-daily dosage of 5 mg to 400 mg.

41. The method according to claim 30, wherein the KCNQ potassium channel opener is Retigabine and is administered in a once-daily dosage of 400 mg.

42. The method according to claim 30, wherein the KCNQ potassium channel opener is Flupirtine and is administered in a once-daily dosage of 400 mg.

43. The method according to claim 30, wherein the subject is a human.

44. The method according to claim 30, wherein an improvement in sleep apnea in a subject is detected by an increase in arterial PCO₂.

45. The method according to claim 30, wherein an improvement in sleep apnea in a subject is detected by an increase in arterial H⁺ concentration.

46. The method according to claim 30, wherein an improvement in sleep apnea in a subject is detected by conducting a sleep study.

47. The method of claim 46, wherein the sleep study assesses at least one of the subject's sleep state, eye movement, muscle activity, heart rate, respiratory effort, airflow, blood oxygen levels, arterial PCO₂, and arterial H⁺ concentration.

48. The method of claim 30, wherein the subject has been diagnosed as suffering from or having a predisposition to a sleep breathing disorder.
